(19) United States
(12) Patent Application Publication
(10) Pub. No.: US 2012/0053218 A1
(43) Pub. Date: Mar. 1, 2012

(54) TRIAZOLE DERIVATIVES AS VASOPRESSIN-RECEPTOR INHIBITORS FOR TREATING CARDIAC INSUFFICIENCY

(75) Inventors: Ulf Brüggemeier, Leichlingen (DE); Chantal Fürstner, Mulheim/Ruhr (DE); Volker Geiß, Ratingen (DE); Joerg Keldenich, Wuppertal (DE); Armin Kern, Wuppertal (DE); Martina Delbeck, Essen (DE); Peter Kolkhof, Wuppertal (DE); Axel Kretschmer, Wuppertal (DE); Elisabeth Pook, Wuppertal (DE); Carsten Schmeck, Mulheim (DE); Hubert Trübel, Wuppertal (DE)

(73) Assignee: BAYER PHARMA AKTIENGESELLSCHAFT, Berlin (DE)

(21) Appl. No.: 13/255,515

(22) PCT Filed: Mar. 9, 2010
(86) PCT No.: PCT/EP2010/001442

§ 371 Date: Nov. 7, 2011

(30) Foreign Application Priority Data
Mar. 18, 2009 (DE) 10 2009 013 642.8

(51) Int. Cl. A61K 31/4196; A61P 7/10; A61P 1/00; A61P 7/00; A61P 1/16; C07D 249/12; A61P 9/04

(52) U.S. Cl. 514/384; 548/263.2

(57) ABSTRACT

The present application relates to novel substituted phenylalanine derivatives, to processes for preparing them, to their use alone or in combinations for the treatment and/or prevention of diseases and also to their use for the production of medicaments for the treatment and/or prevention of diseases, more particularly for the treatment and/or prevention of cardiovascular disorders.

The present application relates to novel substituted phenylalanine derivatives, to processes for preparing them, to their use alone or in combinations for the treatment and/or prevention of diseases and also to their use for the production of medicaments for the treatment and/or prevention of diseases, more particularly for the treatment and/or prevention of cardiovascular disorders.

The liquid content of the human body is subject to various physiological control mechanisms the purpose whereof is to keep it constant (volume homeostasis). In the process, both the volume filling of the vascular system and also the osmolarity of the plasma are continuously recorded by appropriate sensors (baroreceptors and osmoreceptors). The information which these sensors supply to the relevant centers in the brain regulates drinking behavior and controls fluid excretion via the kidneys by means of humoral and neural signals. The peptide hormone vasopressin is of central importance in this [Schrier R. W., Abraham, W. T., New Engl. J. Med. 341, 577-585 (1999)].

Vasopressin is produced in specialized endocrine neurons in the Nucleus supraopticus and N. paraventricularis in the wall of the third ventricle (hypothalamus) and transported from there along its neural processes into the posterior lobes of the hypophysis (neurohypophysis). There the hormone is released into the bloodstream according to stimulus. A loss of volume, e.g. as a result of acute bleeding, heavy sweating, prolonged thirst or diarrhoea, is a stimulus for intensified outpouring of the hormone. Conversely, the secretion of vasopressin is inhibited by an increase in the intravascular volume, e.g. as result of increased fluid intake.

Vasopressin exerts its action mainly via binding to three receptors, which are classified as V1a, V1b and V2 receptors and belong to the family of G protein-coupled receptors. V1a receptors are mainly located on the cells of the vascular smooth musculature. Their activation gives rise to vasoconstriction, as a result of which the peripheral resistance and blood pressure rise. Apart from this, V1a receptors are also detectable in the liver. V1b receptors (also named V3 receptors) are detectable in the central nervous system. Together with corticotropin-releasing hormone (CRH), vasopressin regulates the basal and stress-induced secretion of adrenocorticotropic hormone (ACTH) via the V1b receptor. V2 receptors are located in the distal tubular epithelium and the epithelium of the collecting tubules in the kidney. Their activation renders these epithelia permeable to water. This phenomenon is due to the incorporation of aquaporins (special water channels) in the luminal membrane of the epithelial cells.

The importance of vasopressin for the reabsorption of water from the urine in the kidney becomes clear from the clinical picture of diabetes insipidus, which is caused by a deficiency of the hormone, e.g. owing to hypophysis damage. Patients who suffer from this clinical picture excrete up to 20 liters of urine per 24 hours if they are not given replacement hormone. This volume corresponds to about 10% of the primary urine. Because of its great importance for the reabsorption of water from the urine, vasopressin is also synonymously referred to as antidiuretic hormone (ADH). Logically, pharmacological inhibition of the action of vasopressin/ADH on the V2 receptor results in increased urine excretion. In contrast to the action of other diuretics (thiazides and loop diuretics), however, V2 receptor antagonists cause increased water excretion, without substantially increasing the excretion of electrolytes. This means that by means of V2 antagonist drugs, volume homeostasis can be restored, without in the process affecting electrolyte homeostasis. Hence drugs with V2 antagonist activity appear particularly suitable for the treatment of all disease conditions which are associated with an overloading of the body with water, without the electrolytes being effectively increased in parallel. A significant electrolyte abnormality is measurable in clinical chemistry as hyponatraemia (sodium concentration<135 mmol/L); it is the most important electrolyte abnormality in hospital patients, with an incidence of about 5% or 250 000 cases per year in the USA alone. If the plasma sodium concentration falls below 115 mmol/L, comatose states and death are imminent.

Depending on the underlying cause, a distinction is made between hypovolaemic, euvolaemic and hypervolaemic hyponatraemia. The forms of hypervolaemia with oedema formation are clinically significant. Typical examples of this are the syndrome of inappropriate ADH/vasopressin secretion (SIAD) (e.g. after craniocerebral trauma or as paraneoplasia in carcinomas) and hypervolaemic hyponatraemia in liver cirrhosis, various renal diseases and cardiac insufficiency [De Luca L. et al., Am. J. Cardiol. 96 (suppl.), 19L-23L (2005)]. In particular, patients with cardiac insufficiency, in spite of their relative hyponatraemia and hypervolaemia, often display elevated vasopressin levels, which is seen as the consequence of generally disturbed neurohumoral regulation in cardiac insufficiency [Francis G. S. et al., Circulation 82, 1724-1729 (1990)].

The disturbed neurohormonal regulation essentially manifests itself in an elevation of the sympathetic tone and inappropriate activation of the renin-angiotensin-aldosterone system. While the inhibition of these components by beta receptor blockers on the one hand and by ACE inhibitors or angiotensin receptor blockers on the other is now an inherent part of the pharmacological treatment of cardiac insufficiency, the inappropriate elevation of vasopressin secretion in advanced cardiac insufficiency is at present still not adequately treatable. Apart from the retention of water mediated by V2 receptors and the unfavorable hemodynamic consequences associated therewith in terms of increased backload, the emptying of the left ventricle, the pressure in the pulmonary blood vessels and cardiac output are also adversely affected by V1a-mediated vasoconstriction. Furthermore, on the basis of experimental data in animals, a direct hypertrophy-promoting action on the heart muscle is also attributed to vasopressin. In contrast to the renal effect of volume expansion, which is mediated by activation of V2 receptors, the direct action on the heart muscle is triggered by activation of V1a receptors.

For these reasons, substances which inhibit the action of vasopressin on the V2 and/or on the V1a receptor appear suitable for the treatment of cardiac insufficiency. In particular, compounds with combined activity on both vasopressin receptors (V1a and V2) should both have desirable renal and also hemodynamic effects and thus offer an especially ideal profile for the treatment of patients with cardiac insufficiency. The provision of such combined vasopressin antagonists also appears to make sense inasmuch as a volume diminution mediated solely via V2 receptor blockade can entail the stimulation of osmoreceptors and as a result a further compensatory increase in vasopressin release. As a result, in the absence of a component simultaneously blocking the V1a receptor, the harmful effects of the vasopressin, such as for example vasoconstriction and heart muscle hypertrophy, could be further intensified [Saghi P. et al., Europ. Heart J. 26, 538-543 (2005)].

WO 99/54315 discloses substituted triazolones having neuroprotective action, and WO 2006/117657 describes triazolone derivatives as anti-inflammatory agents. Furthermore, EP 503 548-A1 and EP 587 134-A2 claim cyclic urea derivatives and their use for treating thromboses. Substituted triazolethiones as ion channel modulators are disclosed in WO 2005/097112. WO 2007/134862 describes substituted imidazol-2-ones and 1,2,4-triazolones as vasopressin receptor antagonists for treating cardiovascular disorders.

It is an object of the present invention to provide novel potent selective dual V1a/V2 receptor antagonists which have improved activity at both vasopressin receptors and as such are suitable for the treatment and/or prevention of diseases, more particularly for the treatment and/or prevention of cardiovascular disorders.

The present invention provides compounds of the general formula (I)

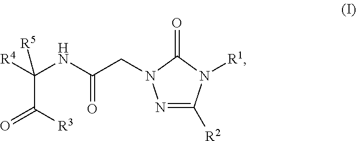

in which
R¹ represents (C₁-C₆)-alkyl, (C₂-C₆)-alkenyl, (C₂-C₆)-alkynyl or (C₃-C₇)-cycloalkyl,
where (C₁-C₆)-alkyl, (C₂-C₆)-alkenyl and (C₂-C₆)-alkynyl may be substituted by 1 to 3 substituents independently of one another selected from the group consisting of halogen, cyano, oxo, hydroxyl, trifluoromethyl, (C₃-C₇)-cycloalkyl, (C₁-C₆)-alkoxy, trifluoromethoxy and phenyl,
where (C₃-C₇)-cycloalkyl may be substituted by 1 or 2 substituents independently of one another selected from the group consisting of (C₁-C₄)-alkyl, oxo, hydroxyl, (C₁-C₄)-alkoxy and amino
and
where (C₁-C₆)-alkoxy may be substituted by 1 or 2 substituents independently of one another selected from the group consisting of amino, hydroxyl, (C₁-C₄)-alkoxy, hydroxycarbonyl and (C₁-C₄)-alkoxycarbonyl
and
where phenyl may be substituted by 1 to 3 substituents independently of one another selected from the group consisting of halogen, cyano, nitro, (C₁-C₄)-alkyl, trifluoromethyl, hydroxyl, hydroxymethyl, (C₁-C₄)-alkoxy, trifluoromethoxy, (C₁-C₄)-alkoxymethyl, hydroxycarbonyl and (C₁-C₄)-alkoxycarbonyl,
and
where (C₃-C₇)-cycloalkyl may be substituted by 1 or 2 substituents independently of one another selected from the group consisting of fluorine, (C₁-C₄)-alkyl, (C₁-C₄)-alkoxy, hydroxyl, amino and oxo,
   R² represents phenyl, naphthyl, thienyl, benzothienyl, furyl or benzofuryl,
where phenyl, naphthyl, thienyl, benzothienyl, furyl and benzofuryl may be substituted by 1 to 3 substituents independently of one another selected from the group consisting of halogen, cyano, nitro, (C₁-C₄)-alkyl, trifluoromethyl, hydroxyl, (C₁-C₄)-alkoxy, trifluoromethoxy and phenyl,
   where phenyl may be substituted by 1 or 2 substituents independently of one another selected from the group consisting of halogen, cyano, nitro, (C₁-C₄)-alkyl, trifluoromethyl, hydroxyl, (C₁-C₄)-alkoxy, trifluoromethoxy, hydroxy-(C₁-C₄)-alkyl and (C₁-C₄)-alkylthio,
      R³ represents hydroxyl or —NR⁶R⁷,
where
R⁶ represents hydrogen or (C₁-C₄)-alkyl,
R⁷ represents hydrogen, (C₁-C₄)-alkyl or (C₃-C₇)-cycloalkyl,
   R⁴ represents phenyl,
where phenyl may be substituted by 1 to 3 substituents independently of one another selected from the group consisting of halogen, cyano, nitro, (C₁-C₄)-alkyl, difluoromethyl, trifluoromethyl, hydroxyl, (C₁-C₄)-alkoxy, difluoromethoxy, trifluoromethoxy and phenyl,
   where phenyl may be substituted by 1 or 2 substituents independently of one another selected from the group consisting of halogen, cyano, nitro, (C₁-C₄)-alkyl, trifluoromethyl, hydroxyl, (C₁-C₄)-alkoxy, trifluoromethoxy, hydroxy-(C₁-C₄)-alkyl and (C₁-C₄)-alkylthio,
      R⁵ represents trifluoromethyl, (C₁-C₄)-alkyl or (C₃-C₇)-cycloalkyl,
      and also their salts, solvates, and solvates of the salts.

Compounds according to the invention are the compounds of the formula (I) and their salts, solvates, and solvates of the salts; the compounds of the below-specified formulae embraced by formula (I), and their salts, solvates, and solvates of the salts; and also the compounds specified below as working examples and embraced by formula (I), and their salts, solvates, and solvates of the salts; in so far as the below-specified compounds embraced by formula (I) are not already salts, solvates, and solvates of the salts.

Depending on their structure, the compounds according to the invention may exist in stereoisomeric forms (enantiomers, diastereomers). The present invention therefore embraces the enantiomers or diastereomers and their respective mixtures. From such mixtures of enantiomers and/or diastereomers it is possible to isolate the stereoisomerically uniform constituents in a known way.

Where the compounds according to the invention are able to occur in tautomeric forms, the present invention embraces all of the tautomeric forms.

Salts preferred in the context of the present invention are physiologically unobjectionable salts of the compounds of the invention. Also embraced are salts which, while not themselves suitable for pharmaceutical applications, may nevertheless be used, for example, for the isolation or purification of the compounds of the invention.

Physiologically acceptable salts of the compounds of the invention embrace acid addition salts of mineral acids, carboxylic acids and sulfonic acids, examples being salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also embrace salts with customary bases, such as—by way of example and preferably—alkali metal salts (e.g. sodium and potassium salts), alkaline earth metal salts (e.g. calcium and magnesium salts) and ammonium salts, derived from ammonia or from organic amines having 1 to 16 C atoms, such as—by way of example and preferably—ethylamine, diethylamine, triethylamine, ethyldiisopropylamine, monoethanolamine, diethanolamine, trisethanolamine, dicyclohexylamine, dimethylaminoethanol, procaine, dibenzylamine, N-methylmorpholine, arginine, lysine, ethylenediamine and N-methylpiperidine.

Solvates in the context of the invention are those forms of the compounds of the invention that form a complex in solid or liquid state by coordination with solvent molecules. Hydrates are one specific form of solvates, where the coordination is with water. Preferred solvates in the context of the present invention are hydrates.

Furthermore, the present invention also embraces prodrugs of the compounds of the invention. The term “prodrugs” embraces compounds which may themselves be biologically active or inactive but which during their residence time in the body are converted (metabolically or by hydrolysis, for example) into compounds of the invention.

In the context of the present invention, the substituents, unless otherwise specified, have the following definitions:

Alkyl in the context of the invention is a straight-chain or branched alkyl radical having 1 to 6 or 1 to 4 carbon atoms. By way of example and for preference it includes the following: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Hydroxyalkyl in the context of the invention is a straight-chain or branched alkyl radical having 1 to 4 carbon atoms which carries a hydroxyl group as substituent in the chain or terminally. By way of example and for preference it includes the following: hydroxymethyl, 1-hydroxyethyl, 2-hydroxyethyl, 1-hydroxy-1-methylethyl, 1,1-dimethyl-2-hydroxyethyl, 1-hydroxypropyl, 2-hydroxypropyl, 3-hydroxypropyl, 1-hydroxy-2-methylpropyl, 2-hydroxy-1-methylpropyl, 2-hydroxy-2-methylpropyl, 1-hydroxybutyl, 2-hydroxybutyl, 3-hydroxybutyl and 4-hydroxybutyl.

Cycloalkyl in the context of the invention is a monocyclic saturated alkyl radical having 3 to 7 or 3 to 6 carbon atoms. By way of example and for preference it includes the following: cyclopropyl, cyclobutyl, cyclopentyl, cyclohexyl and cycloheptyl.

Alkenyl in the context of the invention is a straight-chain or a branched alkenyl radical having 2 to 6 carbon atoms and one or two double bonds. Preference is given to a straight-chain or branched alkenyl radical having 2 to 4 carbon atoms and one double bond. By way of example and for preference it includes the following: vinyl, allyl, isopropenyl and n-but-2-en-1-yl.

Alkynyl in the context of the invention is a straight-chain or branched alkynyl radical having 2 to 6 or 2 to 4 carbon atoms and one triple bond. By way of example and for preference it includes the following: ethynyl, n-prop-1-yn-1-yl, n-prop-2-yn-1-yl, n-but-2-yn-1-yl and n-but-3-yn-1-yl.

Alkoxy in the context of the invention is a straight-chain or branched alkoxy radical having 1 to 6 or 1 to 4 carbon atoms. By way of example and for preference it includes the following: methoxy, ethoxy, n-propoxy, isopropoxy, 1-methylpropoxy, n-butoxy, isobutoxy and tert-butoxy.

Alkylthio in the context of the invention is a thio group having a straight-chain or branched alkyl substituent having 1 to 4 carbon atoms. By way of example and for preference it includes the following: methylthio, ethylthio, n-propylthio, isopropylthio, n-butylthio and tert-butylthio.

Alkoxycarbonyl in the context of the invention is a straight-chain or branched alkoxy radical having 1 to 6 carbon atoms and a carbonyl group attached to the oxygen. By way of example and for preference it includes the following: methoxycarbonyl, ethoxycarbonyl, n-propoxycarbonyl, isopropoxycarbonyl and tert-butoxycarbonyl.

Halogen in the context of the invention includes fluorine, chlorine, bromine and iodine. Preference is given to chlorine or fluorine.

An oxo group in the context of the invention is an oxygen atom attached via a double bond to a carbon atom.

If radicals in the compounds of the invention are substituted, the radicals, unless otherwise specified, may be substituted one or more times. In the context of the present invention it is the case that, for all radicals which occur more than once, their definitions are independent of one another. Substitution by one, two or three identical or different substituents is preferred. Very particular preference is given to substitution by one substituent.

Preference in the context of the present invention is given to compounds of the formula (I) in which

R¹ represents (C₁-C₆)-alkyl, (C₂-C₆)-alkenyl or (C₃-C₆)-cycloalkyl,
where (C₁-C₆)-alkyl and (C₂-C₆)-alkenyl may be substituted by 1 to 3 substituents independently of one another selected from the group consisting of fluorine, chlorine, cyano, oxo, hydroxyl, trifluoromethyl, cyclopropyl, cyclobutyl, methoxy, ethoxy, trifluoromethoxy and phenyl,
   where phenyl may be substituted by 1 to 3 substituents independently of one another selected from the group consisting of fluorine, chlorine, cyano, methyl, ethyl, trifluoromethyl, hydroxyl, hydroxymethyl, methoxy, ethoxy, trifluoromethoxy, methoxymethyl, ethoxymethyl, hydroxycarbonyl, methoxycarbonyl and ethoxycarbonyl,
and
where (C₃-C₆)-cycloalkyl may be substituted by 1 or 2 substituents independently of one another selected from the group consisting of fluorine, methyl, ethyl, methoxy, ethoxy, hydroxyl, amino and oxo,
   R² represents phenyl or thienyl,
where phenyl and thienyl may be substituted by 1 or 2 substituents independently of one another selected from the group consisting of fluorine, chlorine, cyano, methyl, ethyl, trifluoromethyl, hydroxyl, methoxy, ethoxy and trifluormethoxy,
   R³ represents hydroxyl or —NR⁶R⁷,
where
R⁶ represents hydrogen or (C₁-C₄)-alkyl,
R⁷ represents hydrogen, (C₁-C₄)-alkyl or (C₃-C₅)-cycloalkyl,
   R⁴ represents phenyl,
where phenyl may be substituted by 1 to 3 substituents independently of one another selected from the group consisting of fluorine, chlorine, cyano, methyl, ethyl, difluoromethyl, trifluoromethyl, methoxy, ethoxy, difluoromethoxy and trifluoromethoxy,
   R⁵ represents trifluoromethyl, methyl, ethyl, isopropyl or cyclopropyl,
   and also their salts, solvates, and solvates of the salts.

Particular preference in the context of the present invention is given to compounds of the formula (I) in which

R¹ represents (C₁-C₆)-alkyl, (C₂-C₆)-alkenyl or cyclopropyl,
where (C₁-C₆)-alkyl and (C₂-C₆)-alkenyl may be substituted by 1 or 2 substituents independently of one another selected from the group consisting of fluorine, oxo, hydroxyl, trifluoromethyl, cyclopropyl and phenyl,
   where phenyl may be substituted by 1 or 2 substituents independently of one another selected from the group consisting of fluorine, chlorine, methyl and methoxy,
      R² represents phenyl,
where phenyl may be substituted by 1 or 2 substituents independently of one another selected from the group consisting of fluorine, chlorine, methyl, methoxy and trifluoromethoxy,
   R³ represents hydroxyl or —NR⁶R⁷,
where
R⁶ represents hydrogen or methyl,
R⁷ represents hydrogen, methyl or cyclopropyl,
   R⁴ represents phenyl,
where phenyl may be substituted by 1 or 2 substituents independently of one another selected from the group consisting of fluorine, chlorine, methyl, trifluoromethyl, methoxy and trifluoromethoxy,
   R⁵ represents methyl or ethyl,
   and also their salts, solvates, and solvates of the salts.

Particular preference in the context of the present invention is furthermore given to compounds of the formula (I) in which

R¹ represents (C₂-C₄)-alkyl, (C₂-C₄)-alkenyl or cyclopropyl,
where (C₂-C₄)-alkyl and (C₂-C₄)-alkenyl may be substituted by 1 or 2 substituents independently of one another selected from the group consisting of fluorine, oxo, hydroxyl, trifluoromethyl, cyclopropyl and phenyl,
   where phenyl may be substituted by 1 or 2 substituents independently of one another selected from the group consisting of fluorine, chlorine, methyl and methoxy,
      R² represents phenyl,
where phenyl may be substituted by 1 or 2 substituents independently of one another selected from the group consisting of fluorine, chlorine, methyl, methoxy and trifluoromethoxy,
   R³ represents hydroxyl or —NH₂,
   R⁴ represents phenyl,
where phenyl may be substituted by 1 or 2 substituents independently of one another selected from the group consisting of fluorine, chlorine, methyl, trifluoromethyl, methoxy and trifluoromethoxy,
   R⁵ represents methyl or ethyl,
   and also their salts, solvates, and solvates of the salts.

Particular preference in the context of the present invention is furthermore given to compounds of the formula (I) in which
R¹ represents 3,3,3-trifluoroprop-2-en-1-yl, 3,3,3-trifluoropropyl or 1,1,1-trifluoropropan-2-ol-3-yl,
R² represents phenyl,
   where phenyl may be substituted by 1 or 2 substituents independently of one another selected from the group consisting of fluorine, chlorine, methyl, methoxy and trifluoromethoxy,
R³ represents hydroxyl or —NH₂,
R⁴ represents phenyl,
   where phenyl may be substituted by 1 or 2 substituents independently of one another selected from the group consisting of fluorine, chlorine, methyl, trifluoromethyl, methoxy and trifluoromethoxy,
R⁵ represents methyl or ethyl,
   and also their salts, solvates, and solvates of the salts.

Preference in the context of the present invention is also given to compounds of the formula (I) in which R² represents p-chlorophenyl.

Preference in the context of the present invention is also given to compounds of the formula (I) in which R⁵ represents trifluoromethyl, methyl or ethyl,

Preference in the context of the present invention is also given to compounds of the formula (I) in which R³ represents amino.

Preference in the context of the present invention is also given to compounds of the formula (I) in which R³ represents hydroxyl.

Preference in the context of the present invention is also given to compounds of the formula (I) in which

R³ represents —NR⁶R⁷,
where
R⁶ represents hydrogen or methyl,
R⁷ represents hydrogen, methyl or cyclopropyl.

Preference in the context of the present invention is also given to compounds of the formula (I) in which R¹ represents 3,3,3-trifluoroprop-2-en-1-yl.

Preference in the context of the present invention is also given to compounds of the formula (I) in which R¹ represents 3,3,3-trifluoropropyl.

Preference in the context of the present invention is also given to compounds of the formula (I) in which R¹ represents 1,1,1-trifluoropropan-2-ol-3-yl.

Preference in the context of the present invention is also given to compounds of the formula (I) in which

R¹ represents (C₂-C₄)-alkyl or (C₂-C₄)-alkenyl,
where (C₂-C₄)-alkyl and (C₂-C₄)-alkenyl are substituted by 1 or 2 substituents independently of one another selected from the group consisting of fluorine, hydroxyl, oxo and trifluoromethyl.

The radical definitions given individually in the respective combinations and preferred combinations of radicals are also replaced arbitrarily, independently of the particular radical combinations specified, by radical definitions from other combinations.

Very particular preference is given to combinations from two or more of the above-mentioned ranges of preference.

The invention further provides a process for preparing the compounds of the formula (I) according to the invention, characterized in that

[A] a compound of the formula (II)

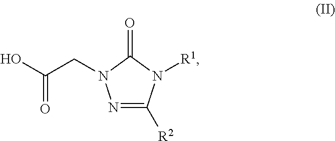

in which R¹ and R² are each as defined above
is coupled in an inert solvent with activation of the carboxylic acid function with a compound of the formula (III)

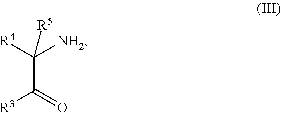

in which R³, R⁴ and R⁵ each have the meanings given above,
   or
   [B] a compound of the formula (IV)

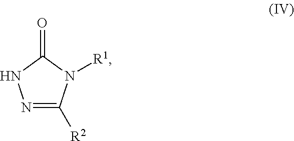

in which R¹ and R² each have the meanings given above,
   is reacted in an inert solvent in the presence of a base with a compound of the formula (V)

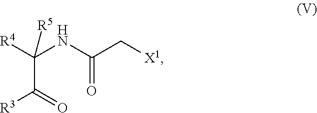

in which R³, R⁴ and R⁵ each have the meanings given above
and
X¹ represents a leaving group such as, for example, halogen, mesylate or tosylate,
   or
   [C] a compound of the formula (I-A)

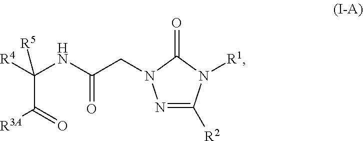

in which R¹, R², R⁴ and R⁵ each have the meanings given above
and
R^{3A} represents hydroxyl,
is reacted in an inert solvent with activation of the carboxylic acid function with an amine of the formula (VI)

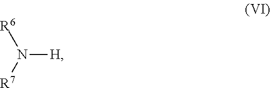

in which R⁶ and R⁷ each have the meanings given above
   and the resulting compounds of the formula (I) are optionally converted with the appropriate (i) solvents and/or (ii) bases or acids into their solvates, salts and/or solvates of the salts.

Inert solvents for the process steps (II)+(III) and (I-A)+(VI)→(I) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or petroleum fractions, halogenated hydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulfoxide, N,N-dimethylformamide, N,N′-dimethylpropyleneurea (DMPU) or N-methylpyrrolidone (NMP). Likewise it is possible to use mixtures of said solvents. Dichloromethane, tetrahydrofuran, dimethylformamide, dimethyl sulfoxide or mixtures of these solvents are preferred.

Suitable condensation agents for the amide formation in process steps (II)+(III) and (I-A)+(VI)→(I) include, for example, carbodiimides such as N,N′-diethyl-, N,N′-dipropyl-, N,N′-diisopropyl- or N,N′-dicyclohexylcarbodiimide (DCC) or N-(3-dimethylaminoisopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium-3 sulfate or 2-tert-butyl-5-methyl-isoxazolium perchlorate, acylamino compounds such as 2-ethoxy-1-ethoxycarbonyl-1,2-dihydroquinoline, or isobutyl chloroformate, propanephosphonic anhydride, diethyl cyanophosphonate, bis-(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with other additives such as 1-hydroxybenzotriazole (HOBt) or N-hydroxysuccinimide (HOSu), and, as bases, alkali metal carbonates, e.g. sodium or potassium carbonate or hydrogen carbonate, or organic bases such as trialkylamines, e.g. triethylamine, N-methylmorpholine, N-methylpiperidine or N,N-diisopropylethylamine. Preferably EDC in combination with HOBt or TBTU in combination with N,N-diisopropylethylamine is used.

The activation of the carboxylic acid function may also be achieved by conversion into the acid chloride, either in situ or as a separate synthesis step. Suitable for this purpose are, for example, sulfonyl chloride or 1-chloro-N,N,2-trimethylprop-1-ene-1-amine.

The condensation (II)+(III) or (I-A)+(VI)→(I) is generally carried out in a temperature range of from −20° C. to +60° C., preferably at from 0° C. to +40° C. The reaction can be carried out at atmospheric, elevated or reduced pressure (for example from 0.5 to 5 bar). The reaction is generally carried out at atmospheric pressure.

Suitable inert solvents for the process step (IV)+(V)→(I) are, for example, halogenated hydrocarbons such as dichloromethane, trichloromethane, carbon tetrachloride, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, dimethyl sulfoxide, N,N′-dimethylpropyleneurea (DMPU), N-methylpyrrolidone (NMP) or pyridine. It is also possible to use mixture of the solvents mentioned. Preference is given to using acetonitrile, acetone or dimethylformamide.

Suitable bases for the process step (IV)+(V)→(I) are the customary inorganic or organic bases. These preferably include alkali metal hydroxides such as, for example, lithium hydroxide, sodium hydroxide or potassium hydroxide, alkali metal or alkaline earth metal carbonates such as lithium carbonate, sodium carbonate, potassium carbonate or cesium carbonate, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preference is given to using potassium carbonate or cesium carbonate.

Here, the base is employed in an amount of from 1 to 5 mol, preferably in an amount from 1 to 2.5 mol, per mole of the compound of the formula (IV). The reaction is generally carried out in a temperature range of from 0° C. to +100° C., preferably at from +20° C. to +80° C. The reaction can be carried out at atmospheric, elevated or reduced pressure (for example from 0.5 to 5 bar). The reaction is generally carried out at atmospheric pressure.

The preparation of the compounds according to the invention can be illustrated by the synthesis schemes below:

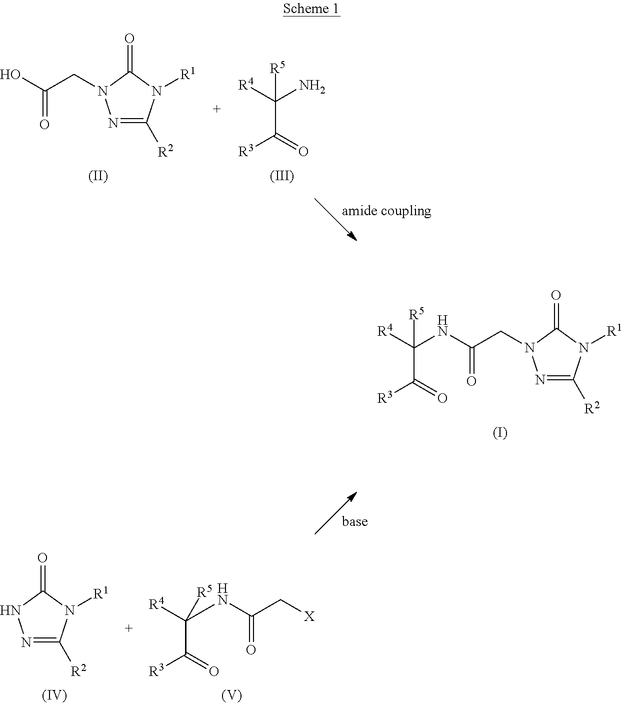

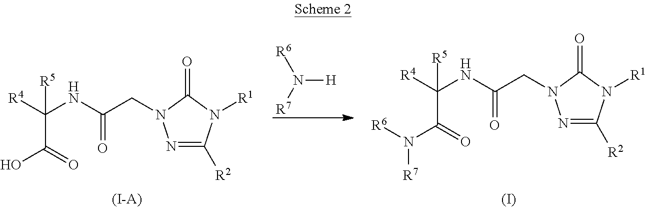

The compounds of the formula (II) can be obtained by base-induced alkylation of 5-aryl-2,4-dihydro-3H-1,2,4-triazol-3-ones of the formula (IV) to give the N²-substituted compounds (VIII) and subsequent ester hydrolysis (see Scheme 3):

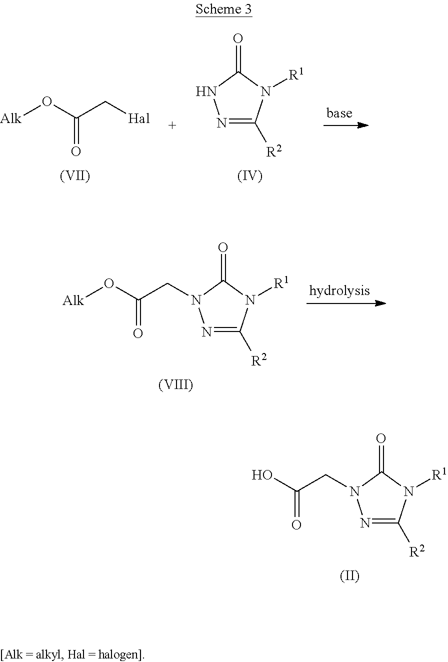

Alternatively, the compounds of the formula (VIII) can also be prepared from N-(alkoxycarbonyl)arylthioamides of the formula (X) known from the literature [see, for example, M. Arnswald, W. P. Neumann, J. Org. Chem. 58 (25), 7022-7028 (1993); E. P. Papadopoulos, J. Org. Chem. 41 (6), 962-965 (1976)] by reaction with hydrazine esters of the formula (IX) and subsequent alkylation at N-4 of the triazolone (XI) (Scheme 4):

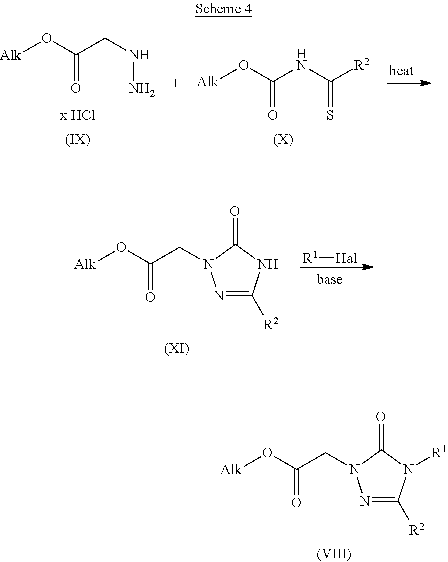

The compounds of the formula (IV) can be prepared from carboxylic acid hydrazides of the formula (XII) by reaction with isocyanates of the formula (XIII) or nitrophenylcarbamates of the formula (XIV) and subsequent base-induced cyclization of the hydrazincarboxamide intermediates (XV) (Scheme 5):

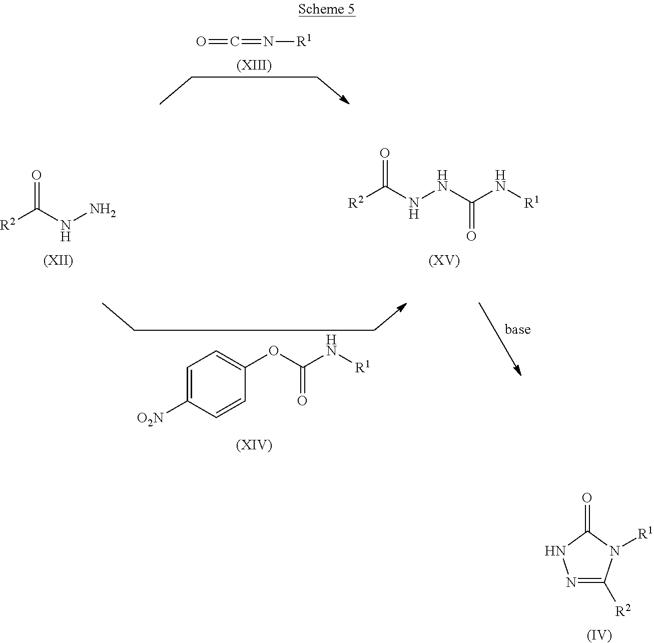

The compound in which R¹ corresponds to the substituent CH₂CH(OH)CF₃ is obtained by, initially following Scheme 4, reacting alkyl isocyanatoacetate (XIIIa) and (XII) to give (XVa). Subsequent basic cyclization affords the triazolone (IVa). The CF₃ group is introduced by reacting (IVa) with trifluoroacetic anhydride in pyridine. The resulting ketone (IVb) can be converted by reduction into (IVc) (Scheme 6):

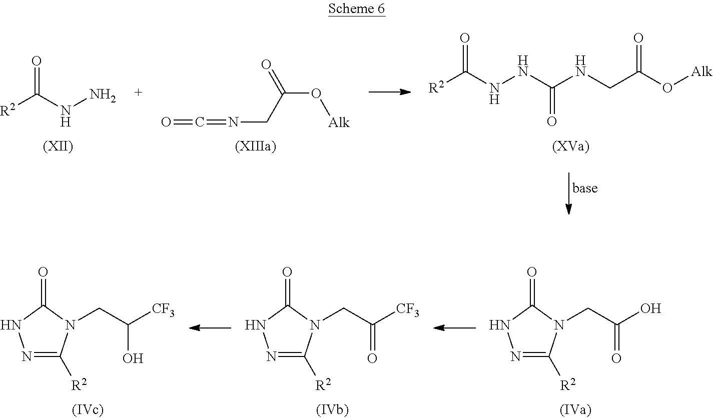

Many of the compounds of the formulae (III), (V), (VI), (VII), (IX), (X), (XII), (XIII), (XIIIa) and (XIV) are commercially available, known from the literature or obtainable by generally known processes.

Further compounds according to the invention may optionally also be prepared by conversions of functional groups of individual substituents, more particularly those set out under R¹, starting from the compounds of the formula (I) obtained by the processes above. These conversions are carried out in accordance with customary methods known to the skilled person, and include, for example, reactions such as nucleophilic and electrophilic substitutions, oxidations, reductions, hydrogenations, transition metal-catalyzed coupling reactions, eliminations, alkylation, amination, esterification, ester cleavage, etherification, ether cleavage, especially formation of carboxamides, and also introduction and removal of temporary protective groups.

The compounds according to the invention possess valuable pharmacological properties and can be used for the prevention and/or treatment of various diseases and disease-induced states in humans and animals.

The compounds according to the invention are potent selective dual V1a/V2 receptor antagonists, which inhibit vasopressin activity in vitro and in vivo and have improved action on both vasopressin receptors.

The compounds according to the invention are particularly suitable for the prophylaxis and/or treatment of cardiovascular diseases. In this connection, the following may for example and preferably be mentioned as target indications: acute and chronic cardiac insufficiency, arterial hypertension, coronary heart disease, stable and unstable angina pectoris, myocardial ischemia, myocardial infarction, shock, arteriosclerosis, atrial and ventricular arrhythmias, transitory and ischemic attacks, stroke, inflammatory cardiovascular diseases, peripheral and cardiac vascular diseases, peripheral circulation disorders, arterial pulmonary hypertension, spasms of the coronary arteries and peripheral arteries, thromboses, thromboembolic diseases, oedema formation such as for example pulmonary oedema, cerebral oedema, renal oedema or cardiac insufficiency-related oedema, and restenoses for example after thrombolysis treatments, percutaneous-transluminal angioplasties (PTA), transluminal coronary angioplasties (PTCA), heart transplants and bypass operations.

In the sense of the present invention, the term cardiac insufficiency also includes more specific or related disease forms such as right cardiac insufficiency, left cardiac insufficiency, global insufficiency, ischemic cardiomyopathy, dilatative cardiomyopathy, congenital heart defects, heart valve defects, cardiac insufficiency with heart valve defects, mitral valve stenosis, mitral valve insufficiency, aortic valve stenosis, aortic valve insufficiency, tricuspidal stenosis, tricuspidal insufficiency, pulmonary valve stenosis, pulmonary valve insufficiency, combined heart valve defects, heart muscle inflammation (myocarditis), chronic myocarditis, acute myocarditis, viral myocarditis, diabetic cardiac insufficiency, alcohol-toxic cardiomyopathy, cardiac storage diseases, diastolic cardiac insufficiency and systolic cardiac insufficiency.

Furthermore, the compounds according to the invention are suitable for use as a diuretic for the treatment of oedemas and in electrolyte disorders, in particular in hypervolaemic and euvolaemic hyponatraemia.

The compounds according to the invention are also suitable for the prophylaxis and/or treatment of polycystic kidney disease (PCKD) and the syndrome of inadequate ADH secretion (SIADH).

In addition, the compounds according to the invention can be used for the prophylaxis and/or treatment of liver cirrhosis, ascites, diabetes mellitus and diabetic complications such as for example neuropathy and nephropathy, acute and chronic kidney failure and chronic renal insufficiency.

Further, the compounds according to the invention are suitable for the prophylaxis and/or treatment of central nervous disorders such as anxiety states and depression, of glaucoma and of cancer, in particular of pulmonary tumors.

Furthermore, the compounds according to the invention can be used for the prophylaxis and/or treatment of inflammatory diseases, asthmatic diseases, chronic-obstructive respiratory tract diseases (COPD), pain conditions, prostatic hypertrophy, incontinence, bladder inflammation, hyperactive bladder, diseases of the adrenals such as for example phaeochromocytoma and adrenal apoplexy, diseases of the intestine such as for example Crohn's disease and diarrhoea, or of menstrual disorders such as for example dysmenorrhoea or of endometriosis.

A further object of the present invention is the use of the compounds according to the invention for the treatment and/or prophylaxis of diseases, in particular of the diseases mentioned above.

A further object of the present invention are the compounds according to the invention for use in a method for the treatment and/or prophylaxis of acute and chronic cardiac insufficiency, hypervolaemic and envolaemic hyponatraemia, liver cirrhosis, ascites, oedemas, and the syndrome of inadequate ADH secretion (SIADH).

A further object of the present invention is the use of the compounds according to the invention for the production of a medicament for the treatment and/or prophylaxis of diseases, in particular of the diseases mentioned above.

A further object of the present invention is a method for the treatment and/or prophylaxis of diseases, in particular of the diseases mentioned above, with the use of an effective quantity of at least one of the compounds according to the invention.

The compounds according to the invention can be used alone or if necessary in combination with other active substances. A further object of the present invention are medicaments which contain at least one of the compounds according to the invention and one or more other active substances, in particular for the treatment and/or prophylaxis of the diseases mentioned above. As combination active substances suitable for this, the following may for example and preferably be mentioned:
organic nitrates and NO donors, such as for example sodium nitroprusside, nitroglycerine, isosorbide mononitrate, isosorbide dinitrate, molsidomine or SIN-1, and inhalational NO;
diuretics, in particular loop diuretics and thiazides and thiazide-like diuretics;
positive-inotropically active compounds, such as for example cardiac glycosides (digoxin), and beta-adrenergic and dopaminergic agonists such as isoproterenol, adrenalin, noradrenalin, dopamine and dobutamine;
compounds which inhibit the degradation of cyclic guanosine monophosphate (cGMP) and/or cyclic adenosine monophosphate (cAMP), such as for example inhibitors of phosphodiesterases (PDE) 1, 2, 3, 4 and/or 5, in particular PDE 5 inhibitors such as sildenafil, vardenafil and tadalafil, and PDE 3 inhibitors such as aminone and milrinone;
natriuretic peptides such as for example “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitizers, such as for example and preferably levosimendan;
NO- and heme-independent activators of guanylate cyclase, such as in particular the compounds described in WO 01/19355, WO 01/19776, WO 01/19778, WO 01/19780, WO 02/070462 and WO 02/070510;
NO-independent, but heme-dependent stimulators of guanylate cyclase, such as in particular riociguat and the compounds described in WO 00/06568, WO 00/06569, WO 02/42301 and WO 03/095451;
inhibitors of human neutrophil elastase (HNE), such as for example sivelestat or DX-890 (reltran);
compounds inhibiting the signal transduction cascade, such as for example tyrosine kinase inhibitors, in particular sorafenib, imatinib, gefitinib and erlotinib;
compounds influencing the energy metabolism of the heart, such as for example and preferably etomoxir, dichloroacetate, ranolazine or trimetazidine;
agents with antithrombotic action, for example and preferably from the group of the thrombocyte aggregation inhibitors, anticoagulants or profibrinolytic substances;
blood pressure-lowering active substances, for example and preferably from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists and rho-kinase inhibitors; and/or
active substances modifying fat metabolism, for example and preferably from the group of the thyroid receptor agonists, cholesterol synthesis inhibitors such as for example and preferably HMG-CoA reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric gallic acid adsorbers, gallic acid reabsorption inhibitors and lipoprotein(a) antagonists.

In a preferred embodiment of the invention, the compounds according to the invention are administered in combination with a diuretic, such as for example and preferably furosemid, bumetanid, torsemid, bendroflumethiazid, chlorthiazid, hydrochlorthiazid, hydroflumethiazid, methyclothiazid, polythiazid, trichlormethiazid, chlorthalidon, indapamid, metolazon, quinethazon, acetazolamid, dichlorophenamid, methazolamid, glycerine, isosorbide, mannitol, amilorid or triamteren.

Agents with antithrombotic action are understood preferably to mean compounds from the group of the thrombocyte aggregation inhibitors, anticoagulants or profibrinolytic substances.

In a preferred embodiment of the invention, the compounds according to the invention are administered in combination with a thrombocyte aggregation inhibitor, such as for example and preferably aspirin, clopidogrel, ticlopidine or dipyridamol.

In a preferred embodiment of the invention, the compounds according to the invention are administered in combination with a thrombin inhibitor, such as for example and preferably ximelagatran, melagatran, bivalirudin or clexane.

In a preferred embodiment of the invention, the compounds according to the invention are administered in combination with a GPIIb/IIIa antagonist, such as for example and preferably tirofiban or abciximab.

In a preferred embodiment of the invention, the compounds according to the invention are administered in combination with a factor Xa inhibitor, such as for example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as for example and preferably coumarin.

Blood pressure-lowering agents are understood preferably to mean compounds from the group of the calcium antagonists, angiotensin AII antagonists, ACE inhibitors, vasopeptidase inhibitors, inhibitors of neutral endopeptidase, endothelin antagonists, renin inhibitors, alpha receptor blockers, beta receptor blockers, mineralocorticoid receptor antagonists, rho-kinase inhibitors and diuretics.

In a preferred embodiment of the invention, the compounds according to the invention are administered in combination with a calcium antagonist, such as for example and preferably nifedipin, amlodipin, verapamil or diltiazem.

In a preferred embodiment of the invention, the compounds according to the invention are administered in combination with an angiotensin AII antagonist, such as for example and preferably losartan, candesartan, valsartan, telmisartan or embusartan.

In a preferred embodiment of the invention, the compounds according to the invention are administered in combination with an ACE inhibitor, such as for example and preferably enalapril, captopril, lisinopril, ramipril, delapril, fosinopril, quinopril, perindopril or trandopril.

In a preferred embodiment of the invention, the compounds according to the invention are administered in combination with a vasopeptidase inhibitor or inhibitor of neutral endopeptidase (NEP).

In a preferred embodiment of the invention, the compounds according to the invention are administered in combination with an endothelin antagonist, such as for example and preferably bosentan, darusentan, ambrisentan or sitaxsentan.

In a preferred embodiment of the invention, the compounds according to the invention are administered in combination with a renin inhibitor, such as for example and preferably aliskiren, SPP-600 or SPP-800.

In a preferred embodiment of the invention, the compounds according to the invention are administered in combination with an alpha-1 receptor blocker, such as for example and preferably prazosin.

In a preferred embodiment of the invention, the compounds according to the invention are administered in combination with a beta receptor blocker, such as for example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with a mineralocorticoid receptor antagonist, such as for example and preferably spironolactone, eplerenon, canrenon or potassium canrenoate.

In a preferred embodiment of the invention, the compounds according to the invention are administered in combination with a rho-kinase inhibitor, such as for example and preferably fasudil, Y-27632, SLx-2119, BF-66851, BF-66852, BF-66853, KI-23095 or BA-1049.

Fat metabolism-modifying agents are understood preferably to mean compounds from the group of the CETP inhibitors, thyroid receptor agonists, cholesterol synthesis inhibitors such as HMG-CoA reductase or squalene synthesis inhibitors, ACAT inhibitors, MTP inhibitors, PPAR-alpha, PPAR-gamma and/or PPAR-delta agonists, cholesterol absorption inhibitors, polymeric gallic acid adsorbers, gallic acid reabsorption inhibitors, lipase inhibitors and lipoprotein(a) antagonists.

In a preferred embodiment of the invention, the compounds according to the invention are administered in combination with a CETP inhibitor, such as for example and preferably dalcetrapib, BAY 60-5521, anacetrapib or CETP-vaccine (CETi-1).

In a preferred embodiment of the invention, the compounds according to the invention are administered in combination with a thyroid receptor agonist, such as for example and preferably D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of the statins, such as for example and preferably lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, such as for example and preferably BMS-188494 or TAK-475.

In a preferred embodiment of the invention, the compounds according to the invention are administered in combination with an ACAT inhibitor, such as for example and preferably avasimibe, melinamide, pactimibe, eflucimibe or SMP-797.

In a preferred embodiment of the invention, the compounds according to the invention are administered in combination with an MTP inhibitor, such as for example and preferably implitapide, BMS-201038, R-103757 or JTT-130.

In a preferred embodiment of the invention, the compounds according to the invention are administered in combination with a PPAR-gamma agonist, such as for example and preferably pioglitazone or rosiglitazone.

In a preferred embodiment of the invention, the compounds according to the invention are administered in combination with a PPAR-delta agonist, such as for example and preferably GW-501516 or BAY 68-5042.

In a preferred embodiment of the invention, the compounds according to the invention are administered in combination with a cholesterol absorption inhibitor, such as for example and preferably ezetimibe, tiqueside or pamaqueside.

In a preferred embodiment of the invention, the compounds according to the invention are administered in combination with a lipase inhibitor, such as for example and preferably orlistat.

In a preferred embodiment of the invention, the compounds according to the invention are administered in combination with a polymeric gallic acid adsorber, such as for example and preferably cholestyramine, colestipol, colesolvam, cholestagel or colestimid.

In a preferred embodiment of the invention, the compounds according to the invention are administered in combination with a gallic acid reabsorption inhibitor, such as for example and preferably ASBT (=IBAT) inhibitors such as for example AZD-7806, S-8921, AK-105, BARI-1741, SC-435 or SC-635.

In a preferred embodiment of the invention, the compounds according to the invention are administered in combination with a lipoprotein(a) antagonist, such as for example and preferably gemcabene calcium (CI-1027) or nicotinic acid.

A further object of the present invention are medicaments which contain at least one compound according to the invention, usually together with one or more inert, non-toxic, pharmaceutically suitable additives, and the use thereof for the aforesaid purposes.

The compounds according to the invention can act systemically and/or locally. For this purpose, they can be administered in a suitable manner, such as for example by the oral, parenteral, pulmonary, nasal, sublingual, lingual, buccal, rectal, dermal, transdermal, conjunctival or otic routes or as an implant or stent.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral or parenteral administration, in particular oral and intravenous administration, are preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as for example ascorbic acid), colorants (e.g. inorganic pigments such as for example iron oxides) and flavor or odor correctors.

In general, to achieve effective results in parenteral administration it has been found advantageous to administer quantities of about 0.001 to 10 mg/kg, preferably about 0.01 to 1 mg/kg body weight. In oral administration, the dosage is about 0.01 bis 100 mg/kg, preferably about 0.01 to 20 mg/kg and quite especially preferably 0.1 to 10 mg/kg body weight.

Nonetheless it can sometimes be necessary to deviate from said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

The following practical examples illustrate the invention. The invention is not limited to the examples.

Unless otherwise stated, the percentages stated in the following tests and examples are percent by weight, parts are parts by weight, and solvent ratios, dilution ratios and concentration information about liquid/liquid solutions are each based on volume.

### A. EXAMPLES

### Abbreviations:

BOC tert-butoxycarbonyl
CI chemical ionization (in MS)
DCI direct chemical ionization (in MS)
DME 1,2-dimethoxyethane
DMF dimethylformamide
DMSO dimethyl sulfoxide
EDC N′-(3-dimethylaminopropyl)-N-ethylcarbodiimide (hydrochloride)
eq. equivalent(s)
ESI electrospray ionization (in MS)
GC/MS gas chromatography-coupled mass spectrometry
sat. saturated
h hour(s)
HOBt 1-hydroxy-1H-benzotriazole hydrate
HPLC high pressure, high performance liquid chromatography
HV high vacuum
conc. concentrated
LC/MS liquid chromatography-coupled mass spectrometry
LDA lithium diisopropylamide
LiHMDS lithium hexamethyldisilazane
min(s) minute(s)
MS mass spectrometry
MTBE methyl tert-butyl ether
NMR nuclear magnetic resonance spectrometry
rac racemic/racemate
R_{f} retention factor (in thin layer chromatography on silica gel)
RT room temperature
Rₜ retention time (in HPLC)
THF tetrahydrofuran
TMOF trimethyl orthoformate
UV ultraviolet spectrometry
v/v volume to volume ratio (of a solution)

### LC/MS, HPLC and GC/MS Methods:

Method 1: MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 2: MS instrument type: Waters (Micromass) Quattro Micro; HPLC instrument type: Agilent 1100 series; column: Thermo Hypersil GOLD 3μ 20×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A (flow 2.5 ml)→5.00 min 100% A; oven: 50° C.; flow rate: 2 ml/min; UV detection: 210 nm.

Method 3: Instrument: Micromass Quattro Premier with Waters HPLC Acquity; column: Thermo Hypersil GOLD 1.9μ 50×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A oven: 50° C.; flow rate: 0.33 ml/min; UV detection: 210 nm.

Method 4: Instrument: Waters ACQUITY SQD HPLC System; column: Waters Acquity HPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A oven: 50° C.; flow rate: 0.40 ml/min; UV detection: 210-400

Method 5: Instrument: Waters ACQUITY SQD HPLC System; column: Waters Acquity HPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A oven: 50° C.; flow rate: 0.40 ml/min; UV detection: 210-400

Method 6: MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 7: MS instrument type: Waters ZQ; HPLC instrument type: Agilent 1100 Series; UV DAD; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.1 min 100% flow rate: 2.5 ml/min, oven: 55° C.; flow rate 2/ml; UV detection: 210 nm.

Method 8 (chiral preparative HPLC): chiral stationary silica gel phase based on the selector poly(N-methacryloyl-D-leucine-dicyclopropylmethylamide); column: 670 mm×40 mm, flow rate: 80 ml/min, temperature: 24° C.; UV detector 260 nM. mobile phase isohexane/ethyl acetate 30:70.
Method 8a: mobile phase: isohexane/ethyl acetate 10:90 (v/v); flow rate: 50 ml/min.

Method 9 (preparative HPLC): chiral stationary silica gel phase based on the selector poly(N-methacryloyl-D-leucine-dicyclopropylmethylamide); column: 250 mm×4.6 mm, mobile phase ethyl acetate 100%, flow rate: 1 ml/min, temperature: 24° C.; UV detector 265 nM.

Method 10 (preparative HPLC): column: Grom-Sil 120 ODS-4HE, 10 μm, SNo. 3331, 250 mm×30 mm. mobile phase A: formic acid 0.1% in water, mobile phase B: acetonitrile; flow rate: 50 ml/min program: 0-3 min: 10% B; 3-27 min: gradient to 95% B; 27-34 min: 95% B; 34.01-38 min: 10% B.

Method 11 (chiral preparative HPLC): stationary phase Daicel Chiralcel OD-H, 5 μm, column: 250 mm×20 mm; temperature: RT; UV detection: 230 nm. Various mobile phases:
Method 11a: mobile phase: isohexane/isopropanol 70:30 (v/v); flow rate: 20 ml/min
Method 11b: mobile phase: isohexane/isopropanol 50:50 (v/v); flow rate: 18 ml/min
Method 11c: mobile phase: isohexane/methanol/ethanol 70:15:15; (v/v/v); flow rate 20 ml/min
Method 11d: mobile phase: isohexane/isopropanol 75:25 (v/v); flow rate 15 ml/min

Method 12 (analytical preparative HPLC): stationary phase Daicel Chiralcel OD-H, column: 250 mm×4 mm; flow rate: 1 ml/min; temperature: RT; UV detection: 230 nm. Various mobile phases:
Method 12a: mobile phase: isohexane/isopropanol 1:1 (v/v);
Method 12b: mobile phase: isohexane/methanol/ethanol 70:15:15 (v/v/v)
Method 12c: mobile phase: isohexane/isopropanol 75:25 (v/v);

Method 13 (chiral preparative HPLC): chiral stationary silica gel phase based on the selector poly-(N-methacryloyl-D-leucine-dicyclopropylmethylamide); column: 600 mm×30 mm, mobile phase: stepped gradient ethyl acetate/methanol 1:1 (0-17 min) ethyl acetate (17.01 min to 21 min)→ethyl acetate/methanol 1:1 (21.01 min to 25 min); flow rate: 80 ml/min, temperature: 24° C.; UV detector 265 nM.

Method 14 (chiral preparative HPLC): as Method 9, but flow rate 2 ml/min.

Method 15 (chiral preparative HPLC): chiral stationary silica gel phase based on the selector poly-(N-methacryloyl-L-isoeucine-3-pentylamide); column: 430 mm×40 mm, flow rate: 80 ml/min, temperature: 24° C.; UV detector 265 nM. Various mobile phases:
Method 15a: 100% ethyl acetate
Method 15b: isohexane/ethyl acetate 10:90

Method 16 (chiral analytical HPLC): chiral stationary silica gel phase based on the selector poly(N-methacryloyl-L-isoeucine-3-pentylamide); column: 250 mm×4.6 mm, mobile phase 100% EA, flow rate 2 ml/min, temperature 24° C.; UV detector 265 nM.

Method 17 (chiral preparative HPLC): chiral stationary silica gel phase based on the selector poly-(N-methacryloyl-L-leucine-(+)-3-pinanemethylamide); column: 600 mm×30 mm, flow rate: 80 ml/min, temperature: 24° C.; UV detector 265 nM. Various mobile phases:
Method 17a: isohexane/ethyl acetate 20:80
Method 17b: isohexane/ethyl acetate 30:70
Method 17c: isohexane/ethyl acetate 50:50
Method 17d: 100% ethyl acetate
Method 17e: isohexane/ethyl acetate 40:60
Method 17f: isohexane/ethyl acetate 10:90

Method 18 (chiral analytical HPLC): chiral stationary silica gel phase based on the selector poly(N-methacryloyl-L-leucine-(+)-3-pinanemethylamide); column: 250 mm×4.6 mm, temperature 24° C.; UV detector 265 nM.
Method 18a: mobile phase: isohexane/ethyl acetate 50:50, flow rate: 2 ml/min.
Method 18b: mobile phase: 100% ethyl acetate, flow rate: 2 ml/min.
Method 18c: mobile phase: 100% ethyl acetate, flow rate: 1 ml/min.

Method 19 (preparative HPLC): column Grom-Sil 1200DS-4HE 10 μm, 250 mm×30 mm; mobile phase: A=water, B=acetonitrile; gradient: 0.0 min 10% B, 3 min 10% B, 30 min 95% B, 42 min 95% B, 42.01 min 10% B, 45 min 10% B; flow rate: 50 ml/min; column temperature: RT; UV detection: 210 nm.

### Starting Materials and Intermediates

### Example 1A

### Ethyl N-({2-[(4-chlorophenyl)carbonyl]hydrazinyl}carbonyl)glycinate

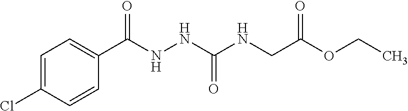

A suspension of 12.95 g (75.9 mmol) of 4-chlorobenzohydrazide in 50 ml of dry THF was initially charged at 50° C., and a solution of 10.0 g (77.5 mmol) of ethyl 2-isocyanatoacetate in 100 ml of dry THF was added dropwise. Initially, a solution was formed, and then a precipitate. After the addition had ended, the mixture was stirred at 50° C. for another 2 h and then allowed to stand at RT overnight. The crystals were isolated by filtration, washed with a little diethyl ether and dried under HV. This gave 21.43 g (89% of theory) of the title compound.

LC/MS [Method 1]: Rₜ=1.13 min.; m/z=300 (M+H)⁺

¹H NMR (DMSO-d₆, 400 MHz): δ [ppm]=10.29 (s, 1H), 8.21 (s, 1H), 7.91 (d, 2H), 7.57 (d, 2H), 6.88 (br.s, 1H), 4.09 (q, 2H), 3.77 (d, 2H), 1.19 (t, 3H)

### Example 2A

### [3-(4-Chlorophenyl)-5-oxo-1,5-dihydro-4H-1,2,4-triazol-4-yl]acetic acid

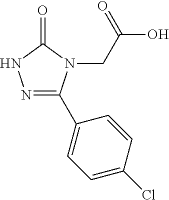

91 ml of a 3N aqueous sodium hydroxide solution were added to 21.43 g (67.93 mmol) of the compound from Example 1A, and the mixture was heated at reflux overnight. After cooling to RT, the mixture was adjusted to pH 1 by slowly adding about 20% strength hydrochloric acid. The precipitated solid was isolated by filtration, washed with water and dried at 60° C. under reduced pressure. Yield: 17.55 g (90% of theory, purity about 88%) of the title compound.

LC/MS [Method 1]: Rₜ=0.94 min.; m/z=254 (M+H)⁺

¹H NMR (DMSO-d₆, 400 MHz): δ [ppm]=13.25 (br.s, 1H), 12.09 (s, 1H), 7.65-7.56 (m, 4H), 4.45 (s, 2H).

### Example 3A

### 5-(4-Chlorophenyl)-4-(3,3,3-trifluoro-2-oxopropyl)-2,4-dihydro-3H-1,2,4-triazol-3-one (or as hydrate: 5-(4-chlorophenyl)-4-(3,3,3-trifluoro-2,2-dihydroxypropyl)-2,4-dihydro-3H-1,2,4-triazol-3-one)

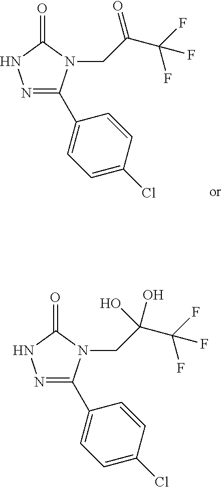

Under argon, 5 g (16.36 mmol) of the compound from Example 2A were dissolved in 200 ml of pyridine, and 17.18 g (81.8 mmol) of trifluoroacetic anhydride were then added. During the addition, the temperature increased to about 35° C. After 30 min the pyridine was removed on a rotary evaporator and the residue was diluted with 1.5 l of 0.5N hydrochloric acid. This mixture was heated to 70° C. and then filtered while still hot. The solid was washed with a little water. The entire filtrate was extracted three times with ethyl acetate. The combined organic phases were washed with water, then with a saturated aqueous sodium bicarbonate solution and then with a saturated aqueous sodium chloride solution, dried over sodium sulfate and freed from the solvent on a rotary evaporator. The residue was dried under HV. Yield: 3.56 g (68% of theory) of the title compound as hydrate.

LC/MS [Method 1]: Rₜ=1.51 min.; m/z=306 (M+H)⁺ and 324 (M+H)⁺ (ketone and hydrate, respectively)

¹H NMR (DMSO-d₆, 400 MHz): δ [ppm]=12.44 (s, 1H), 7.72 (d, 2H), 7.68 (br.s, 2H), 7.61 (d, 2H), 3.98 (s, 2H).

### Example 4A

### 5-(4-Chlorophenyl)-4-(3,3,3-trifluoro-2-hydroxypropyl)-2,4-dihydro-3H-1,2,4-triazol-3-one

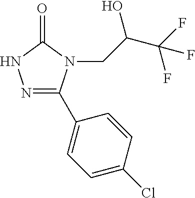

3.56 g (11 mmol) of the compound from Example 3A were dissolved in 100 ml of methanol, and 3.75 g of sodium borohydride (99 mmol) were added with ice-cooling (evolution of gas). After 1.5 h, 200 ml of 1M hydrochloric acid were added slowly. The methanol was removed on a rotary evaporator and the residue was diluted with 500 ml of water and extracted three times with ethyl acetate. The combined organic phases were washed with a saturated aqueous sodium bicarbonate solution and then with a saturated aqueous sodium chloride solution, dried over sodium sulfate and freed from the solvent on a rotary evaporator. The residue was dried under HV. This gave 3.04 g (90% of theory) of the title compound.

LC/MS [Method 2]: Rₜ=1.80 min.; m/z=308 (M+H)⁺.

¹H NMR (DMSO-d₆, 400 MHz): δ [ppm]=12.11 (s, 1H), 7.75 (d, 2H), 7.62 (d, 2H), 6.85 (d, 1H), 4.34-4.23 (m, 1H), 3.92 (dd, 1H), 3.77 (dd, 1H).

### Example 5A

### Methyl {3-(4-chlorophenyl)-5-oxo-4-(3,3,3-trifluoro-2-hydroxypropyl)-4,5-dihydro-1H-1,2,4-triazol-1-yl}acetate

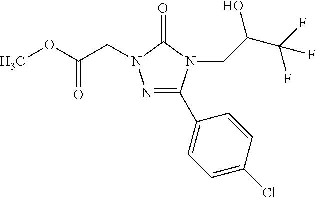

3.04 g (9.9 mmol) of the compound from Example 4A were dissolved in 100 ml of acetonitrile, and 1.07 g (9.9 mmol) of methyl chloroacetate, 2.73 g (19.8 mmol) of potassium carbonate and a small spatula tip of potassium iodide was added. The reaction mixture was heated at reflux for 1 h, allowed to cool to RT and filtered. The filtrate was freed from the volatile components on a rotary evaporator and the residue was dried under HV. Yield: 3.70 g (89% of theory, purity 90%) of the title compound.

LC/MS [Method 3]: Rₜ=1.10 min.; m/z=380 (M+H)⁺.

¹H NMR (DMSO-d₆, 400 MHz): δ [ppm]=7.78 (d, 2H), 7.64 (d, 2H), 6.91 (d, 1H), 4.72 (s, 2H), 4.16-4.35 (m, 1H), 3.99 (dd, 1H), 3.84 (dd, 1H), 3.70 (s, 3H).

The racemic compound from Example 5A could be separated into its enantiomers Example 6A and Example 7A by preparative HPLC on a chiral phase, as described in WO 2007/134862.

Column: chiral silica gel phase based on the selector poly(N-methacryloyl-L-isoleucine-3-pentylamide, 430 mm×40 mm; mobile phase: stepped gradient isohexane/ethyl acetate 1:1→ethyl acetate→isohexane/ethyl acetate 1:1; flow rate: 50 ml/min; temperature: 24° C.; UV detection: 260 nm.

This gave, from 3.6 g of the racemic compound from Example 5A (dissolved in 27 ml of ethyl acetate and 27 ml of I isohexane and separated on the column in three portions), 1.6 g of enantiomer 1 (Example 6A), which eluted first, and also 1.6 g of enantiomer 2 (Example 7A), which eluted later.

### Example 6A

### Methyl {3-(4-chlorophenyl)-5-oxo-4-[(2S)-3,3,3-trifluoro-2-hydroxypropyl]-4,5-dihydro-1H-1,2,4-triazol-1-yl}acetate

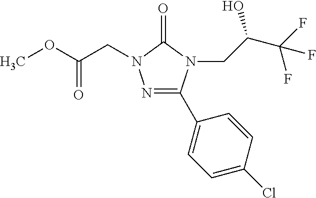

The enantiomer that eluted first in the racemate separation of Example 5A.

Rₜ=3.21 min [column: chiral silica gel phase based on the selector poly(N-methacryloyl-L-isoleucine-3-pentylamide, 250 mm×4.6 mm; mobile phase: isohexane/ethyl acetate 1:1; flow rate: 1 ml/min; UV detection: 260 nm].

### Example 7A

### Methyl {3-(4-chlorophenyl)-5-oxo-4-[(2R)-3,3,3-trifluoro-2-hydroxypropyl]-4,5-dihydro-1H-1,2,4-triazol-1-yl}acetate

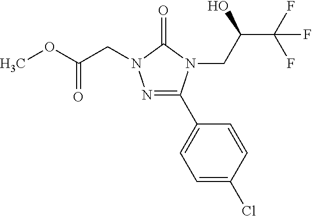

The enantiomer that eluted last in the racemate separation of Example 5A.

Rₜ=4.48 min [column: chiral silica gel phase based on the selector poly(N-methacryloyl-L-isoleucine-3-pentylamide, 250 mm×4.6 mm; mobile phase: isohexane/ethyl acetate 1:1; flow rate: 1 ml/min; UV detection: 260 nm].

### Example 8A

### {3-(4-Chlorophenyl)-5-oxo-4-[(2S)-3,3,3-trifluoro-2-hydroxypropyl]-4,5-dihydro-1H-1,2,4-triazol-1-yl}acetic acid

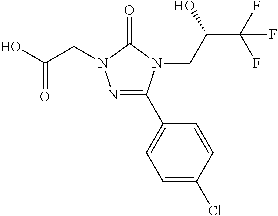

The enantiomerically pure ester from Example 6A (1.6 g, 4.21 mmol) was dissolved in 77 ml of methanol, and 17 ml of a 1M solution of lithium hydroxide in water were added. The mixture was stirred at RT for 1 h and then freed from methanol on a rotary evaporator. The residue was diluted with 100 ml of water and acidified with 1 N of hydrochloric acid to pH 1-2. The precipitated product was filtered off, washed successively with water and cyclohexane and filtered. Drying under HV gave the title compound (1.1 g, 71% of theory).

[α]_{D}²⁰=+3.4° (methanol, c=0.37 g/100 ml)

LC/MS [Method 1]: Rₜ=1.51 min; m/z=366 (M+H)⁺

¹H-NMR (400 MHz, DMSO-d₆): δ [ppm]=3.84 (dd, 1H), 4.00 (dd, 1H), 4.25 (m, 1H), 4.58 (s, 2H), 6.91 (d, 1H), 7.63 (d, 2H), 7.78 (d, 2H), 13.20 (br. s, 1H).

### Example 9A

### {3-(4-Chlorophenyl)-5-oxo-4-[(2R)-3,3,3-trifluoro-2-hydroxypropyl]-4,5-dihydro-1H-1,2,4-triazol-1-yl}acetic acid

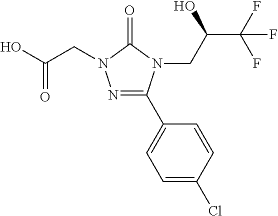

Analogously to Example 8A, Example 7A gave the title compound.

[α]_{D}²⁰=−4.6° (methanol, c=0.44 g/100 ml)

LC/MS [Method 1]: Rₜ=1.53 min; m/z=366 (M+H)⁺

¹H-NMR (400 MHz, DMSO-d₆): δ [ppm]=3.84 (dd, 1H), 4.00 (dd, 1H), 4.25 (m, 1H), 4.58 (s, 2H), 6.91 (d, 1H), 7.63 (d, 2H), 7.78 (d, 2H), 13.20 (br. s, 1H).

### Example 10A

Methyl {3-(4-chlorophenyl)-5-oxo-4-[(1E)-3,3,3-trifluoroprop-1-en-1-yl]-4,5-dihydro-1H-1,2,4-triazol-1-yl}acetate

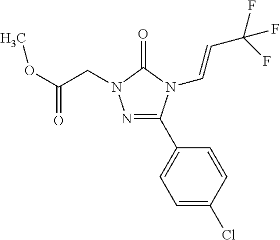

At RT, 280 mg (0.74 mmol) of the compound from Example 7A together with 108.1 mg (0.89 mmol) of 4-dimethylaminopyridine were initially charged in 5.3 ml of pyridine, 0.31 ml (1.84 mmol) of trifluoromethanesulfonic anhydride were added a little at a time and the mixture was stirred for 12 h. The pyridine was removed on a rotary evaporator. The residue was taken up in acetonitrile and 1N hydrochloric acid and purified by preparative HLPC (Method 10). This gave 230 mg (86% of theory) of the title compound.

LC/MS [Method 4]: Rₜ=1.14 min; m/z=362 (M+H)⁺

¹H-NMR (400 MHz, DMSO-d₆): δ [ppm]=3.72 (s, 3H), 4.78 (s, 2H), 6.85 (dd, 1H), 7.18 (d, 1H), 7.68 (s, 4H).

### Example 11A

### {3-(4-Chlorophenyl)-5-oxo-4-[(1E)-3,3,3-trifluoroprop-1-en-1-yl]-4,5-dihydro-1H-1,2,4-triazol-1-yl}acetic acid

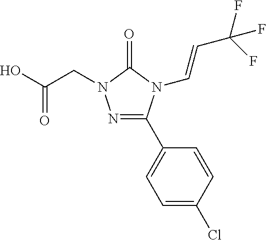

260 mg (0.72 mmol) of the compound from Example 10A were dissolved in 5 ml of methanol, and 2.87 ml (2.87 mmol) of a 1 M solution of lithium hydroxide in water were added. The mixture was stirred at RT for 1 h and then acidified with 1 N hydrochloric acid and diluted with DMSO. The entire solution was purified by preparative HLPC (Method 10). This gave 215 mg (86% of theory) of the title compound.

LC/MS [Method 4]: Rₜ=1.03 min.; m/z=348 (M+H)⁺

¹H-NMR (400 MHz, DMSO-d₆): δ [ppm]=4.64 (s, 2H), 6.79-6.92 (m, 1H), 7.19 (dd, 1H), 7.68 (s, 4H), 13.31 (br. s, 1H).

### Example 12A

### 2-Amino-2-[3-(trifluoromethyl)phenyl]propanamide

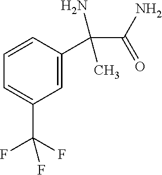

138 ml of water, 108 ml of 25% strength aqueous ammonia solution and 173 ml of ethanol were initially charged, and 108 g (574.0 mmol) of 1-[3-(trifluoromethyl)phenyl]ethanone, 30 g (574 mmol) of sodium cyanide and 31 g (631 mmol) of ammonium chloride were then added.

This mixture was stirred in an autoclave at 70° C. for 20 h. The ethanol was removed on a rotary evaporator and the residue was extracted 4× with in each case 500 ml of ether. Magnesium sulfate and activated carbon were added to the combined organic phases, and the mixture was filtered off with suction through kieselguhr. The filtrate was concentrated on a rotary evaporator. The residue was then purified by chromatography on 2 kg of silica gel 60 (mobile phase: cyclohexane/ethyl acetate 3:1 to 1:1).

With ice-cooling, 500 ml of concentrated hydrochloric acid were added slowly to the intermediate 2-amino-2-[3-(trifluoromethyl)phenyl]propionitrile (56 g, 46% of theory) isolated in this manner. The suspension was stirred at RT overnight. On a rotary evaporator, the volume was reduced to 150 ml. 250 ml of acetone were added, and all volatile components were removed on a rotary evaporator. With ice-cooling, 125 ml of concentrated aqueous ammonia solution were added to the solid paste that remained. The mixture was stirred in the ice-bath for 30 minutes. The crystals were filtered off with suction and washed 2× with in each case 50 ml of ice-water, and then with pentane. The product was dried under high vacuum. This gave 43 g (32% of theory) of the title compound.

MS (ESIpos): m/z=233 [M+H]⁺.

H-NMR (400 MHz, CDCl₃): δ [ppm]=1.82 (s, 3H), 5.54 (br.s, 1H), 7.26 (br.s, 1H), 7.48 (t, 1H), 7.55 (d, 2H), 7.75 (d, 1H), 7.83 (s, 1H).

### Example 13A

### tert-Butyl {1-amino-1-oxo-2-[3-(trifluoromethyl)phenyl]propan-2-yl}carbamate

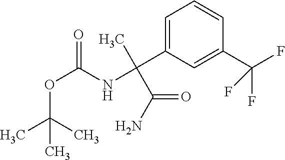

At RT, 43.0 g (185 mmol) of 2-amino-2-[3-(trifluoromethyl)phenyl]propanamide together with 53.6 g (638 mmol) of sodium bicarbonate were initially charged in 245 ml of DMF and 245 ml of tert-butanol, and 99.5 g (456 mmol) of di-tert-butyl dicarbonate were then added. The mixture was stirred at 60° C. for 3 days. For work-up, the mixture was diluted with ethyl acetate and washed successively twice with water, twice with 1M hydrochloric acid and once with saturated aqueous sodium chloride solution, The organic phase was dried over sodium sulfate and concentrated under reduced pressure. The residue was taken up in DMSO and separated by preparative HPLC (Method 7). The product fraction was concentrated on a rotary evaporator. The residue was dried under high vacuum. This gave 30.0 g (50% of theory) of the title compound.

LC/MS [Method 2]: Rₜ=2.11 min; m/z=333 (M+H)⁺

¹H-NMR (400 MHz, DMSO-d₆): δ [ppm]=1.82 (s, 3H), 7.09 (br.s, 1H), 7.27-7.40 (m, 2H), 7.53-7.65 (m, 2H), 7.65-7.73 (m, 2H).

The two enantiomers could be separated by HPLC on a chiral phase [Method 13]: see Examples 14A and 15A.

### Example 14A

### tert-Butyl {(2R)-1-amino-1-oxo-2-[3-(trifluoromethyl)phenyl]propan-2-yl}carbamate (Enantiomer I)

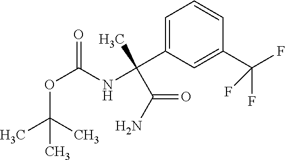

The enantiomer that eluted first (12.1 g) in the enantiomer separation according to Method 13 of 21.5 g of the compound from Example 13A.

Chiral analytical HPLC [Method 14]: Rₜ=2.89 min.

### Example 15A

### tert-Butyl {(2S)-1-amino-1-oxo-2-[3-(trifluoromethyl)phenyl]propan-2-yl}carbamate (Enantiomer II)

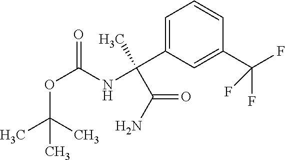

The enantiomer that eluted last (12.1 g) in the enantiomer separation according to Method 13 of 21.5 g of the compound from Example 13A.

Chiral analytical HPLC [Method 14]: Rₜ=4.55 min.

### Example 16A

### (2R)-2-Amino-2-[3-(trifluoromethyl)phenyl]propanamide hydrochloride

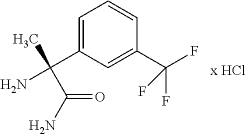

At RT, 12 g (36.1 mmol) of tert-butyl {(2R)-1-amino-1-oxo-2-[3-(trifluoromethyl)phenyl]propan-2-yl}carbamate from Example 14A were pre-dissolved in 20 ml of dichloromethane, 50 ml of a 4M solution of hydrogen chloride in dioxane were then added and the mixture was stirred for 1 h. The mixture was concentrated under reduced pressure and the residue was dried under high vacuum. 100 ml of dichloromethane were added to the residue, and the mixture was kept in an ultrasonic bath for 10 minutes. The solid was filtered off with suction, washed with a little dichloromethane and dried under high vacuum. This gave 8.14 g (84% of theory) of the title compound.

LC/MS [Method 2]: Rₜ=0.51 min; m/z=233 (M+H)⁺

¹H-NMR (400 MHz, DMSO-d₆): δ [ppm]=1.95 (s, 3H), 7.69-7.94 (m, 6H), 8.85 (br.s, 3H).

### Example 17A

### (2S)-2-Amino-2-[3-(trifluoromethyl)phenyl]propanamide hydrochloride

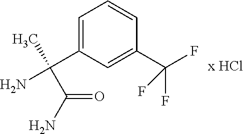

At RT, 11.5 g (34.6 mmol) of tert-butyl {(2S)-1-amino-1-oxo-2-[3-(trifluoromethyl)phenyl]propan-2-yl}carbamate from Example 15A were pre-dissolved in 20 ml of dichloromethane, a 4M solution of hydrogen chloride in dioxane was then added and the mixture was stirred for 1 h. Under reduced pressure, the mixture was concentrated to ⅓ of the original volume, when the product precipitated in crystalline form. The mixture was diluted with 100 ml of dichloromethane and kept in an ultrasonic bath for 10 minutes. The solid was filtered off with suction, washed with a little dichloromethane and dried under high vacuum. This gave 7.56 g (82% of theory) of the title compound.

LC/MS [Method 2]: Rₜ=0.55 min; m/z=233 (M+H)⁺

¹H-NMR (400 MHz, DMSO-d₆): δ [ppm]=1.94 (s, 3H), 7.67-7.80 (m, 3H), 7.80-7.91 (m, 3H), 8.79 (br.s, 3H).

### Example 18A

### 2-Amino-2-[2-fluoro-3-(trifluoromethyl)phenyl]propanamide hydrochloride

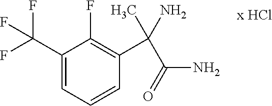

At 70° C., 5 g (24.3 mmol) of 1-[2-fluoro-3-(trifluoromethyl)phenyl]ethanone, 1.248 g (25.5 mmol) of sodium cyanide, 1.427 g (26.7 mmol) of ammonium chloride and 3.6 ml of 25% strength aqueous ammonia solution were stirred together in 6 ml of water and 7.5 ml of ethanol for 17 h. The dark-brown solution was cooled to RT and concentrated on a rotary evaporator to ⅓ of the original volume. The residue was extracted 3× with diethyl ether. Magnesium sulfate and activated carbon were added to the combined organic phases, and the mixture was stirred for 30 min and then filtered. 8 ml of a 4M solution of hydrogen chloride in dioxane were added to the filtrate, and the mixture was stirred for 5 min and freed from the volatile components on a rotary evaporator. 20 ml of concentrated hydrochloric acid were added to the residue, and the mixture was stirred overnight. The mixture was diluted with water to 300 ml and extracted 3× with in each case 50 ml of dichloromethane. The aqueous phase was made alkaline with 35% strength aqueous ammonia solution (pH about 9-10) and extracted 3× with in each case 75 ml of dichloromethane. The combined organic phases were dried over sodium sulfate and concentrated under reduced pressure. The residue was taken up in 150 ml of diethyl ether, and 8 ml of a 4M solution of hydrogen chloride in dioxane were added. The mixture was concentrated under reduced pressure and dried under high vacuum. This gave 1.97 g (24% of theory, purity 86%) of the title compound.

LC/MS [Method 3]: Rₜ=0.25 min; m/z=251 (M+H)⁺

¹H-NMR (400 MHz, DMSO-d₆): δ [ppm]=1.93 (s, 3H), 7.49-7.77 (m, 3H), 7.84-8.04 (m, 2H), 8.74 (br.s, 3H).

### Example 19A

### 2-Amino-2-[3-(trifluoromethyl)phenyl]butanamide hydrochloride

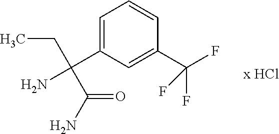

At 70° C., 9.8 g (48.5 mmol) of 1-[3-(trifluoromethyl)phenyl]propan-1-one, 3.8 g (77.6 mmol) of sodium cyanide, 4.4 g (82.4 mmol) of ammonium chloride and 10 ml of 35% strength aqueous ammonia solution were stirred together in 25 ml of water and 30 ml of ethanol for 17 h. The solution was cooled to RT. On a rotary evaporator, the volume was reduced to ⅓ of the original volume. The residue was extracted 3× with diethyl ether. Magnesium sulfate and activated carbon were added to the combined organic phases, and the mixture was stirred for 30 minutes and then filtered. 20 ml of a 4M solution of hydrogen chloride in dioxane were added to the filtrate, and the precipitated solid was filtered off with suction. 40 ml of concentrated hydrochloric acid were added to the solid, and the mixture was stirred overnight. The mixture was diluted with water to 300 ml and washed 3× with in each case 50 ml of dichloromethane. The aqueous phase was made alkaline with 35% strength aqueous ammonia solution (pH about 9-10) and extracted 3× with in each case 75 ml of dichloromethane. The combined organic phases were dried over sodium sulfate, 10 ml of a 4M solution of hydrogen chloride in dioxane were then added and the mixture was freed from the solvent on a rotary evaporator. The solid was dried under high vacuum and then re-dissolved in water and purified by preparative HPLC (Method 7). The product fraction was freed from the solvent on a rotary evaporator and then dried under high vacuum. This gave 190 mg (1.4% of theory) of the title compound.

LC/MS [Method 2]: Rₜ=0.78 min; m/z=247 (M+H)⁺

¹H-NMR (400 MHz, DMSO-d₆): δ [ppm]=0.76 (t, 3H), 1.79-1.93 (dq, 1H), 1.97-2.10 (dq, 1H), 7.11 (br.s, 1H), 7.38 (br.s, 1H), 7.51-7.62 (m, 2H), 7.82 (d, 1H), 7.87 (s, 1H).

### Example 20A

### 2-Amino-2-cyclopropyl-2-[3-(trifluoromethyl)phenyl]acetamide hydrochloride

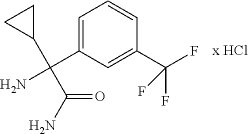

At 70° C., 1.6 g (7.5 mmol) of cyclopropyl[3-(trifluoromethyl)phenyl]methanone, 384 mg (7.8 mmol) of sodium cyanide, 440 mg (8.2 mmol) of ammonium chloride and 1 ml of 35% strength ammonia solution were stirred together in 3 ml of water and 3 ml of ethanol for 17 h. The solution was cooled to RT and, on a rotary evaporator, reduced to ⅓ of the original volume. The residue was extracted 3× with diethyl ether. Magnesium sulfate and activated carbon were added to the combined organic phases, and the mixture was stirred for 30 min and then filtered. 10 ml of a 4M solution of hydrogen chloride in dioxane were added to the filtrate, and the mixture was concentrated under reduced pressure. 20 ml of concentrated hydrochloric acid were added to the residue, and the mixture was stirred overnight. The mixture was diluted with water to 100 ml and washed 3× with in each case 50 ml of dichloromethane. The aqueous phase was made alkaline with 35% strength aqueous ammonia solution (pH about 9-10) and extracted three times with in each case 75 ml of dichloromethane. The combined organic phases were dried over sodium sulfate, and 10 ml of a 4M solution of hydrogen chloride in dioxane were added. The mixture was freed from all volatile components on a rotary evaporator. The residue was dried under high vacuum and then re-dissolved in water and purified by preparative HPLC (Method 10). The product fraction was freed from the solvent on a rotary evaporator and then dried under high vacuum. This gave 24 mg (1% of theory) of the title compound of a purity of about 80%.

LC/MS [Method 2]: Rₜ=0.95 min; m/z=259 (M+H)⁺

¹H-NMR (400 MHz, DMSO-d₆): δ [ppm]=0.54-0.67 (m, 1H), 0.74 (m, 2H), 0.80-0.98 (m, 1H), 1.68-1.87 (m, 1H), 7.54 (s, 1H), 7.73-7.80 (m, 1H), 7.82-7.97 (m, 4H), 8.46-8.72 (m, 3H).

### Example 21A

### tert-Butyl [(2R)-1-amino-2-(2-chlorophenyl)-1-oxopropan-2-yl]carbamate

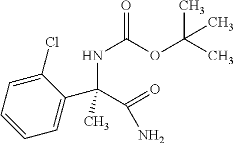

500 mg (2.12 mmol) of (2R)-2-amino-2-(2-chlorophenyl)propanoic acid hydrochloride (from Netchem, New Brunswick N.J. 08901, USA, Article No.: 506093-HCl) were dissolved in 10 ml of 10% strength aqueous sodium bicarbonate solution. 10 ml of dioxane and 511 μl (2.22 mmol) of di-tert-butyl dicarbonate were added, and the reaction mixture was stirred at RT overnight. By addition of 1N hydrochloric acid, the pH was adjusted to 2, and the product was then extracted three times with ethyl acetate. The combined organic phases were dried over sodium sulfate and the solvent was removed on a rotary evaporator. The residue was dried under HV and corresponds to the intermediate (2R)-2-[(tert-butoxycarbonyl)amino]-2-(2-chlorophenyl)propanoic acid (322 mg, 51% of theory LC-MS [Method 3]: Rₜ=1.08 min. m/z: ES pos.: 322 (M+Na)⁺, ES neg.: 298 (M−H)⁻.

100 mg (0.334 mmol) of the (2R)-2-[(tert-butoxycarbonyl)amino]-2-(2-chlorophenyl)propanoic acid obtained in this manner and 81 mg (0.6 mmol) of HOBt were initially charged in 3 ml of dimethylformamide, 115 mg (0.6 mmol) of EDC were added and the reaction mixture was stirred at RT for 20 minutes. 2 ml of 32% strength aqueous ammonia solution were then added, and the mixture was stirred at RT overnight. The mixture was adjusted to pH 2 with 1N hydrochloric acid and separated by preparative HPLC (Method 10). The product fraction was freed from the solvent on a rotary evaporator and then dried under high vacuum. This gave 59 mg (59% of theory) of the title compound.

LC/MS [Method 3]: Rₜ=1.02 min; m/z=299 [M+H]⁺.

¹H-NMR (400 MHz, DMSO-d₆): δ [ppm]=1.26 (br.s, 7H), 1.84 (s, 3H), 6.46-6.70 (m, 1H), 6.85 (br.s, 1H), 7.25-7.44 (m, 4H), 7.64 (d, 1H).

### Example 22A

### (2R)-2-Amino-2-(2-chlorophenyl)propanamide hydrochloride

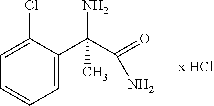

2 ml of dichloromethane and 2 ml of a 4M solution of hydrogen chloride in dioxane were added to 58 mg (0.194 mmol) of tert-butyl [(2R)-1-amino-2-(2-chlorophenyl)-1-oxopropan-2-yl]carbamate from Example 21A, and the mixture was stirred at RT for 2 h. All volatile components were removed on a rotary evaporator, and the white solid was dried under high vacuum. This gave 50 mg (46% of theory) of the title compound.

LC/MS [Method 2]: Rₜ=0.22 min; m/z=199 (M+H)⁺

¹H-NMR (400 MHz, DMSO-d₆): δ [ppm]=1.89 (s, 3H), 7.30 (s, 1H), 7.47-7.57 (m, 3H), 7.61 (s, 1H), 7.68-7.77 (m, 1H), 8.40 (br.s, 3H).

### Example 23A

### 5-Methyl-5-[3-(trifluoromethyl)phenyl]imidazolidine-2,4-dione (Racemate)

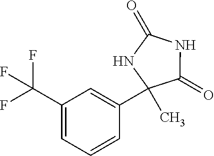

A mixture of 25 g (133 mmol) of 3-trifluoromethylacetophenone, 10.4 g (159 mmol) of potassium cyanide and 63.8 g (664 mmol) of ammonium carbonate in 300 ml of water and 300 ml of ethanol was stirred at 60° C. overnight. The ethanol was removed on a rotary evaporator. The product precipitated from the aqueous mixture that remained. The product was filtered off, washed three times with water and dried under HV. This gave 31 g (90% of theory) of the title compound.

LC/MS [Method 3]: Rₜ=0.90 min; m/z=259 (M+H)+

¹H-NMR (400 MHz, DMSO-d6): δ [ppm]=1.69 (s, 3H), 7.62-7.70 (m, 1H), 7.71-7.76 (m, 1H), 7.78 (s, 1H), 7.83 (d, 1H), 8.75 (s, 1H), 10.91 (br.s, 1H).

The two enantiomers could be separated by chromatography on a chiral phase (Method 8): see Examples 24A and 25A.

### Example 24A

### (5R)-5-Methyl-5-[3-(trifluoromethyl)phenyl]imidazolidine-2,4-dione

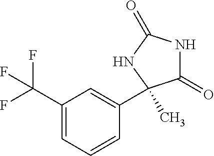

The enantiomer that eluted first (14.6 g) in the separation according to Method 8 of 30.3 g of the compound from Example 23A.

Chiral analytical HPLC [Method 9]: Rₜ=2.9 min.

[α]_{D}²⁰=−102.2° (methanol, c=0.53 g/100 ml)

The absolute configuration was determined by hydrolysis to Example 26A and comparison with the commercial amino acid (see Example 27A).

### Example 25A

### (5S)-5-Methyl-5-[3-(trifluoromethyl)phenyl]imidazolidine-2,4-dione

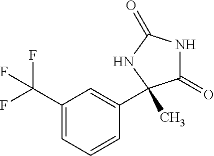

The enantiomer that eluted last (13.8 g) in the separation according to Method 8 of 30.3 g of the compound from Example 23A.

Chiral analytical HPLC [Method 9]: Rₜ=5.4 min.

[α]_{D}²⁰=+102.4° (methanol, c=0.53 g/100 ml)

### Example 26A

### 2-Amino-2-[3-(trifluoromethyl)phenyl]propanoic acid (Racemate)

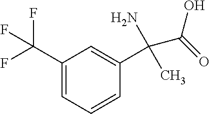

300 mg (1.16 mmol) of the compound from Example 23A were heated under reflux in 3 ml of 1N aqueous sodium hydroxide solution for 3 days. After cooling to RT, the reaction mixture was acidified (pH 1-2) by careful addition of 6N hydrochloric acid. During the addition, some of the product precipitated as a gel. The mixture was diluted with 200 ml of water and washed twice with ethyl acetate. The aqueous phase was freed from the water on a rotary evaporator. The residue was stirred in methanol, and the resulting suspension was filtered. The filtrate was freed from the methanol on a rotary evaporator. The residue was dissolved in a 2:1 mixture of acetonitrile/water and purified by preparative HPLC (Method 10). This gave 205 mg (76% of theory) of the title compound.

LC/MS [Method 3]: Rₜ=0.34 min; m/z=234 (M+H)⁺

¹H-NMR (400 MHz, DMSO-d₆): δ [ppm]=1.67 (s, 3H), 7.56-7.68 (m, 2H), 7.79 (d, 1H), 7.86 (s, 1H), 8.20 (br.s, 2H).

### Example 27A

### (2R)-2-Amino-2-[3-(trifluoromethyl)phenyl]propanoic acid

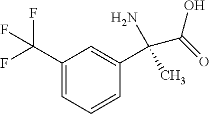

In 400 ml of 2N aqueous sodium hydroxide solution, 14.6 g (56.7 mmol) of the compound from Example 24A were heated at reflux for 23 h. The reaction mixture was cooled to 0° C. (ice bath) and 6N hydrochloric acid was added slowly to pH 1. The precipitated solid was filtered off. The filtrate was concentrated to dryness on a rotary evaporator. The residue was stirred in 300 ml of methanol, and the resulting suspension was filtered. The filtrate was concentrated on a rotary evaporator. The residue was taken up in water and purified by preparative HPLC (Method 7). The product obtained was dried under HV (12 g, 91% of theory)

LC/MS [Method 3]: Rₜ=0.33 min; m/z=234 (M+H)⁺

[α]_{D}²⁰=−44.1° (methanol, c=0.50 g/100 ml).

¹H-NMR (400 MHz, DMSO-d₆): δ [ppm]=1.67 (s, 3H), 7.55-7.68 (m, 2H), 7.79 (d, 1H), 7.86 (s, 1H), 8.19 (br.s, 3H).

75 mg of this amino acid were treated with an excess of a 4N solution of hydrogen chloride in dioxane, freed from the volatile components on a rotary evaporator and dried under HV. The resulting hydrochloride shows the following analytical data:

[α]_{D}²⁰=−63.8° (methanol, c=0.51 g/100 ml).

¹H-NMR (400 MHz, DMSO-d₆): δ [ppm]=1.91 (s, 3H), 7.75 (t, 1H), 7.84 (d, 1H), 7.86-7.94 (m, 2H), 9.18 (br.s, 3H).

The optical rotation is comparable to the optical rotation determined for the commercial (2R)-2-amino-2-[3-(trifluoromethyl)phenyl]propanoic acid hydrochloride (Netchem, New Brunswick N.J. 08901, USA, Article No.: 506085-HCl): [α]_{D}²⁰=−44.1° (methanol, c=0.50 g/100 ml). Accordingly, the (R) configuration was recorded for Example 24A and for Example 26A, and the (S) configuration for Example 25A and Example 27A.

### Example 28A

### (2S)-2-Amino-2-[3-(trifluoromethyl)phenyl]propanoic acid

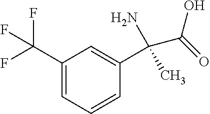

Analogously to Example 26A, hydrolysis of 13.1 g (50.7 mmol) of the compound from Example 25A gave 8.22 g (69% of theory) of the title compound.

LC/MS [Method 2]: Rₜ=0.92 min; m/z=234 (M+H)⁺

[α]_{D}²⁰=+47.0° (methanol, c=0.50 g/100 ml).

¹H-NMR (400 MHz, DMSO-d₆): δ [ppm]=1.67 (s, 3H), 7.55-7.68 (m, 2H), 7.79 (d, 1H), 7.86 (s, 1H), 8.19 (br.s, 3H).

This amino acid in acetonitrile was treated with an excess of a 1N solution of hydrochloric acid. The volatile components were then removed on a rotary evaporator and the residue was dried under HV. The resulting hydrochloride shows the following analytical data:

[α]_{D}²⁰=−63.8° (methanol, c=0.51 g/100 ml).

¹H-NMR (400 MHz, DMSO-d₆): δ [ppm]=1.91 (s, 3H), 7.75 (t, 1H), 7.84 (d, 1H), 7.86-7.94 (m, 2H), 9.18 (br.s, 3H).

### Example 29A

### 2-Amino-3,3,3-trifluoro-2-[3-(trifluoromethyl)phenyl]propanoic acid (Mixture of Enantiomers)

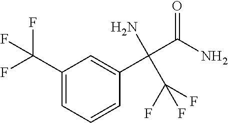

Analogously to H. Wang et al., Organic Letters 2006, 8 (7), 1379-1381, 2.50 g (10.3 mmol) of 2,2,2-trifluoroacetophenone and 2.50 g (20.7 mmol) of (R)-tert-butylsulfinamide were initially charged in 21 ml of n-hexane, and 4.40 g (4.57 ml, 15.5 mmol) of titanium(IV) isopropoxide were added. The reaction mixture was stirred at RT overnight, and the reaction was then stopped by addition of 9 ml of water with ice-bath cooling. After 5 min, the entire mixture was filtered through Celite. The filtrate was concentrated on a rotary evaporator. The residue (2.86 g) was dissolved in 17 ml of n-hexane, and 1.66 ml of (12.4 mmol) of trimethylsilyl cyanide were added at RT. The reaction mixture was stirred at RT for three days, 30 ml of 10% strength aqueous ammonium chloride solution were then added and the mixture was extracted three times with ethyl acetate. The combined organic phases were dried over sodium sulfate and freed from the solvent using a rotary evaporator. Without purification and analysis, the residue (3.04 g) was reacted further. To this end, the entire amount was, with ice-cooling, dissolved in 23 ml of conc. sulfuric acid and then stirred at RT for 3 h. The reaction mixture was poured onto ice and extracted three times with ethyl acetate. The combined organic phases were dried over sodium sulfate and concentrated on a rotary evaporator. This gave residue A. The acidic aqueous phase was adjusted to pH 7 using 20% strength aqueous sodium hydroxide solution and three more times extracted with ethyl acetate. The combined organic phases were dried over sodium sulfate and concentrated on a rotary evaporator. This gave residue B. The two residues A and B were combined and separated by preparative HPLC (Method 10). The appropriate fraction was concentrated on a rotary evaporator and the aqueous phase that remained was adjusted to pH 14 with 2M aqueous sodium hydroxide solution and then extracted with dichloromethane three times. The combined dichloromethane phases were dried over sodium sulfate and concentrated on a rotary evaporator. The oil corresponded to the title compound (136 mg, 5% of theory).

¹H-NMR (400 MHz, DMSO-d6): δ [ppm]=7.49 (br.s, 1H), 7.59 (br.s, 1H), 7.67 (t, 1H), 7.78 (d, 1H), 7.97 (d, 1H), 8.02 (s, 1H).

### Example 30A

### {4-[(2S)-2-{[tert-Butyl(dimethyl)silyl]oxy}-3,3,3-trifluoropropyl]-3-(4-chlorophenyl)-5-oxo-4,5-dihydro-1H-1,2,4-triazol-1-yl}acetic acid

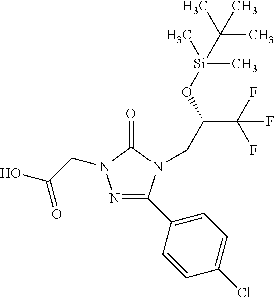

2.46 ml (1.5 eq) of a solution comprising 0.5M of tert-butyldimethylsilyl chloride and 1 M of imidazole in DMF were added to 300 mg (0.82 mmol) of the compound from Example 8A. The reaction mixture was stirred at RT overnight. Another 1.5 eq. of the above solution were then added, and the mixture was stirred for 24 h. This procedure was repeated until a total of 6 eq. of tert-butyldimethylsilyl chloride had been added. 6 ml of a 2M aqueous sodium carbonate solution were then added, and the reaction mixture was stirred for 30 min. The pH was adjusted to 4 by addition of 1M hydrochloric acid and the mixture was extracted three times with dichloromethane. The combined organic phases were washed with water and then dried over sodium sulfate and concentrated on a rotary evaporator. The residue was dried under HV. This gave the title compound: 407 mg (93% of theory).

LC/MS [Method 5]: Rₜ=1.33 min; m/z=480 (M+H)⁺

¹H-NMR (400 MHz, DMSO-d₆): δ [ppm]=−0.11 (s, 3H), 0.06 (s, 3H), 0.80 (s, 9H), 4.01 (dd, 1H), 4.13 (dd, 1H), 4.54-4.63 (m, 1H), 4.60 (s, 2H), 7.69 (d, 2H), 7.80 (d, 2H).

### Example 31A

### N-Cyclopropyl-2-{[2-(trifluoromethoxy)phenyl]carbonyl}hydrazinecarboxamide

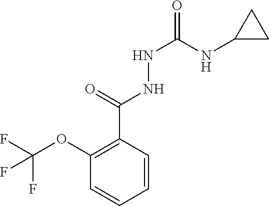

At 60° C., 2.00 g (9.09 mmol) of 2-trifluoromethoxybenzhydrazide were dissolved in dry THF (50 ml), and 0.79 g (9.09 mmol) of cyclopropyl isocyanate dissolved in 10 ml of dry tetrahydrofuran was then added dropwise. The mixture was stirred at 60° C. for 18 h. After cooling to RT, the mixture was stirred with about 50 ml of diethyl ether. The colorless solid was filtered off with suction, washed with diethyl ether and dried under high vacuum. This gave 2.57 g (93% of theory) of the target compound.

LC-MS [Method 6] Rₜ=1.43 min; MS [ESIpos]: m/z=304 (M+H)⁺.

¹H-NMR (400 MHz, CDCl₃): δ [ppm]=0.61-0.69 (m, 2H), 0.77-0.85 (m, 2H), 2.60-2.68 (m, 1H), 5.45 (br.s, 1H), 7.34 (d, 1H), 7.42 (t, 1H), 7.52-7.62 (m, 2H), 7.99 (dd, 1H), 8.63 (br.s, 1H).

### Example 32A

### 4-Cyclopropyl-5-[2-(trifluoromethoxy)phenyl]-2,4-dihydro-3H-1,2,4-triazol-3-one

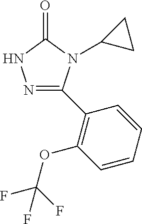

2.53 g (8.3 mmol) of the compound from Example 31A were suspended in 15 ml of 3M aqueous sodium hydroxide solution and heated at reflux for 96 h. After cooling, the pH was adjusted to 10 using semiconcentrated hydrochloric acid. The precipitated solid was filtered off with suction, washed with water until neutral and then stirred with methanol. The mixture was filtered, the filtrate was concentrated on a rotary evaporator and the residue was dried under high vacuum. This gave 1.81 g (55% of theory, purity 72%) of the desired compound which was directly reacted further as such.

LC-MS [Method 6] Rₜ=1.76 min; MS [ESIpos]: m/z=286 (M+H)⁺.

### Example 33A

### Methyl {4-cyclopropyl-5-oxo-3-[2-(trifluoromethoxy)phenyl]-4,5-dihydro-1H-1,2,4-triazol-1-yl}acetate

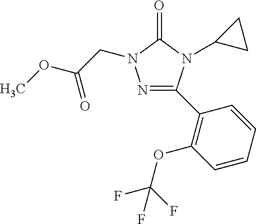

1.81 g (4.60 mmol) of the compound from Example 32A were dissolved in 15 ml of acetonitrile, and 1.64 g of cesium carbonate (5.03 mmol) were added. 0.48 ml (5.48 mmol) of methyl chloro-acetate was then added at RT. The mixture was heated under reflux for 2 h and then, at RT, diluted with 20 ml of ethyl acetate and washed with 10 ml of water. The aqueous phase was extracted two more times with in each case 10 ml of ethyl acetate, and the extracts were dried over magnesium sulfate, filtered and concentrated under reduced pressure. This gave 1.46 mg (82% of theory) of the target compound.

LC-MS [Method 3] Rₜ=1.05 min; MS [ESIpos]: m/z=358 (M+H)⁺.

¹H-NMR (400 MHz, CDCl₃): δ[ppm]=0.58-0.66 (m, 2H), 0.78-0.85 (m, 2H), 2.95 (spt, 1H), 3.78 (s, 3H), 4.64 (s, 2H), 7.37-7.45 (m, 2H), 7.53-7.63 (m, 2H).

### Example 34A

### {4-Cyclopropyl-5-oxo-3-[2-(trifluoromethoxy)phenyl]-4,5-dihydro-1H-1,2,4-triazol-1-yl}acetic acid

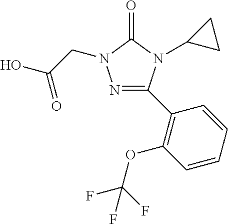

1.46 g (4.09 mmol) of the compound from Example 33A were dissolved in 8 ml of methanol, and 4.9 ml (4.9 mmol) of a 1N solution of lithium hydroxide were added at RT. After 30 min, the solvent was removed under reduced pressure and the residue was taken up in 20 ml of water and 20 ml of ethyl acetate. After phase separation, the aqueous phase was acidified with 1N hydrochloric acid and extracted twice with in each case 15 ml of ethyl acetate. The combined organic phases were dried over magnesium sulfate, filtered and concentrated under reduced pressure, and the residue was dried under high vacuum. This gave 1.25 g (85% of theory) of the target compound, which was reacted further without further purification.

LC-MS [Method 6] Rₜ=1.82 min; MS [ESIpos]: m/z=344 (M+H)⁺.

¹H-NMR (400 MHz, CDCl₃): δ[ppm]=0.60-0.66 (m, 2H), 0.77-0.86 (m, 2H), 2.96 (spt, 1H), 4.67 (s, 2H), 7.37-7.45 (m, 2H), 7.55-7.63 (m, 2H).

### Working Examples

### Example 1

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanoic acid (Diastereomer Mixture)

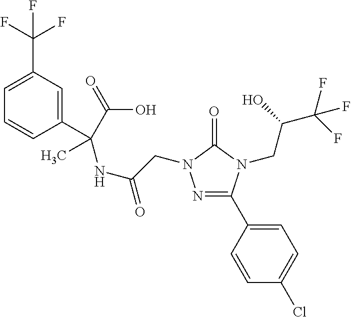

At RT, 56 mg of the compound from Example 8A (0.15 mmol), 29 mg (0.15 mmol) of EDC and 21 mg (0.15 mmol) of HOBt were stirred in 2.2 ml of DMF for 20 min, and 50 mg (0.18 mmol) of the compound from Example 26A and 53 μl (0.31 mmol) of N,N-diisopropylethylamine were then added. The mixture was stirred at RT for 20 min, 1 ml of 1N hydrochloric acid was then added and the complete mixture was separated by preparative HPLC (Method 10). This gave 54 mg (61% of theory) of the title compound.

LC-MS [Method 6]: Rₜ=2.78 min; MS [ESIpos]: m/z=581 (M+H)⁺

¹H-NMR (400 MHz, DMSO-d₆): δ [ppm]=1.85 (s, 3H), 3.82 (dd, 1H), 3.96 (br.d, 1H), 4.19-4.35 (m, 1H), 4.58 (s, 2H), 6.92 (d, 1H), 7.54-7.70 (m, 4H), 7.71-7.82 (m, 4H), 8.80 (s, 1H), 13.11 (br.s, 1H).

### Example 2

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanamide (Diastereomer Mixture)

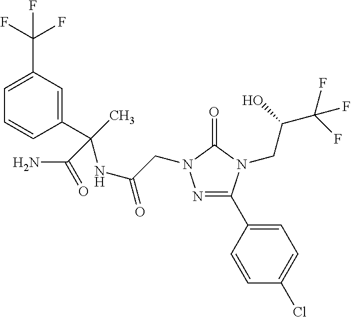

54 mg of the compound from Example 1 (90 μmol) and 24 mg (179 μmol) of HOBt were initially charged in 1.3 ml of DMF, and 34 mg (179 μmol) of EDC were added. The mixture was stirred at RT for 20 min, 5 ml of an ammonia solution (35% in water) were then added and the mixture was stirred for another 20 min. 1 ml of 1N hydrochloric acid was added, and the complete mixture was separated by preparative HPLC (Method 10). The appropriate fraction was freed from the solvents on a rotary evaporator and the residue was dried under HV. This gave 49 mg (94% of theory) of the title compound.

LC-MS [Method 6]: Rₜ=2.28 min; MS [ESIpos]: m/z=580 (M+H)⁺

¹H-NMR (400 MHz, DMSO-d₆): δ [ppm]=1.88 (d (1 s per diastereomer, 3H), 3.74-3.89 (dd, 1H), 3.94 (dd, 1H), 4.26 (m, 1H), 4.48-4.69 (m, 2H), 6.90 (t (1 d per diastereomer), 1H), 7.33 (br.s, 1H), 7.41 (br.d (1 br.s per diastereomer), 1H), 7.52-7.69 (m, 4H), 7.68-7.83 (m, 4H), 8.63 (s, 1H).

The diastereomers from Example 2 could be separated by preparative chromatography on a chiral phase (Method 17b): see Example 3 and Example 4.

### Example 3

### (2S)-2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanamide

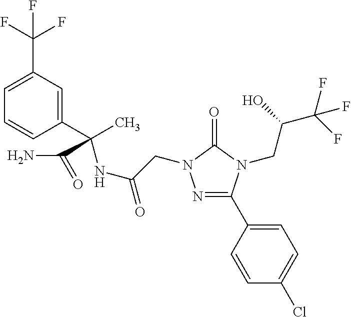

Initially eluted diastereomer (19 mg) from the separation of 49 mg of the compound from Example 2 according to Method 17b.

Chiral analytical HPLC (Method 18b): Rₜ=1.73 min.

Alternatively, the title compound can be prepared by the process below:

3.50 g of the compound from Example 8A (9.57 mmol) and 2.04 g (14.36 mmol) of HOBt were initially charged in 100 ml of DMF, and 2.75 g (14.36 mmol) of EDC were added. The mixture was stirred at RT for 15 min, and 2.83 g (10.5 mmol) of the compound from Example 17A and 2.0 ml (11.5 mmol) of N,N-diisopropylethylamine were then added. The reaction mixture was stirred at RT overnight and then diluted with 1 l of water and extracted three times with in each case 400 ml of ethyl acetate. The combined organic phases were washed successively twice with 1N hydrochloric acid, once with water, twice with a saturated aqueous sodium bicarbonate solution and once with a saturated aqueous sodium chloride solution, then dried over sodium sulfate and freed from the solvent using a rotary evaporator. The residue was purified by preparative HPLC (Method 10). This gave 4.09 g (74% of theory) of the title compound.

LC-MS [Method 3]: Rₜ=1.20 min; MS [ESIpos]: m/z=580 (M+H)⁺

¹H-NMR (400 MHz, DMSO-d6): δ [ppm]=1.88 (s, 3H), 3.82 (dd, 1H), 3.91-4.01 (m, 1H), 4.26 (br.s, 1H), 4.50-4.63 (m [AB], 2H), 6.91 (d, 1H), 7.33 (s, 1H), 7.42 (s, 1H), 7.54-7.60 (m, 1H), 7.60-7.66 (m, 3H), 7.69-7.80 (m, 4H), 8.63 (s, 1H).

### Example 4

### (2R)-2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanamide

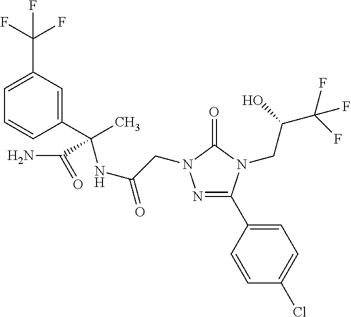

Last eluted diastereomer (16 mg) from the separation of 49 mg of the compound from Example 2 according to Method 17b.

Chiral analytical HPLC (Method 18b): Rₜ=2.45 min.

Alternatively, the title compound can be prepared by the process below (A):

6.00 g of the compound from Example 8A (16.4 mmol) and 3.32 g (24.6 mmol) of HOBt were initially charged in 160 ml of DMF, and 4.72 g (24.6 mmol) of EDC were added. The mixture was stirred at RT for 15 min, and 4.85 g (18.0 mmol) of the compound from Example 16A and 3.4 ml (19.7 mmol) of N,N-diisopropylethylamine were then added. The reaction mixture was stirred at RT overnight and then diluted with 1.2 l of water and extracted three times with in each case 400 ml of ethyl acetate. The combined organic phases were washed successively twice with 1N hydrochloric acid, once with water, twice with a saturated aqueous sodium bicarbonate solution and once with a saturated aqueous sodium chloride solution, then dried over sodium sulfate and freed from the solvent using a rotary evaporator. The residue was purified by preparative HPLC (Method 10). This gave 6.67 g (70% of theory) of the title compound.

LC-MS [Method 3]: Rₜ=1.22 min; MS [ESIpos]: m/z=580 (M+H)⁺

¹H-NMR (400 MHz, DMSO-d₆): δ [ppm]=1.88 (s, 3H), 3.78-3.87 (m, 1H), 3.92-3.99 (m, 1H), 4.26 (br.s, 1H), 4.53-4.63 (m [AB], 2H), 6.90 (d, 1H), 7.33 (s, 1H), 7.41 (s, 1H), 7.53-7.60 (m, 1H), 7.60-7.66 (m, 3H), 7.68-7.79 (m, 4H), 8.64 (s, 1H).

Alternatively, the title compound can be prepared by the process below (B):

1.80 g (4.92 mmol) of the compound from Example 8A (4.92 mmol) and 700 mg (5.41 mmol) of HOBt were initially charged in 30 ml of DMF, and 944 mg (5.41 mmol) of EDC were added. The mixture was stirred at RT for 20 min and then added dropwise to a suspension of 1.46 g (5.41 mmol) of the compound from Example 27A and 1.03 ml (5.91 mmol) of N,N-diisopropylethylamine in 30 ml of DMF. The reaction mixture was stirred at RT for 1 h and then diluted with 500 ml of 0.5N hydrochloric acid and extracted three times with ethyl acetate. The combined organic phases were washed three times with water and then once with a saturated aqueous sodium chloride solution and dried over sodium sulfate. The solvent was removed on a rotary evaporator. The residue was dried under HV. The product obtained in this manner (3.44 g), which corresponds to the compound from Example 6 in a purity of about 70% (4.21 mmol), was reacted further without purification: the total amount and 1.02 g (7.57 mmol) of HOBt were dissolved in 40 ml of DMF, and 1.45 g (7.57 mmol) of EDC were then added. The solution obtained in this manner was stirred at RT for 30 min and then added dropwise to an ammonia solution (35% in water, 45 ml) which had been initially charged. This mixture was stirred for 20 min and then concentrated on a rotary evaporator. 500 ml of water were added to the residue. The solution was extracted three times with in each case 250 ml of ethyl acetate. The combined organic phases were washed successively three times with 1N hydrochloric acid, once with water, twice with a saturated aqueous sodium bicarbonate solution and once with a saturated aqueous sodium chloride solution, then dried over sodium sulfate and freed from the solvent using a rotary evaporator. The residue was purified by preparative HPLC (Method 7). This gave 2.30 g (3.97 mmol, 80% of theory) of the title compound.

### Example 5

### (2R)-2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]propanoic acid

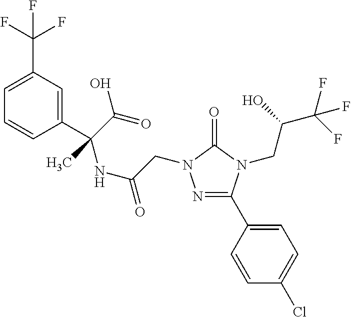

250 mg of the compound from Example 8A (0.68 mmol) and 92 mg (0.68 mmol) of HOBt were initially charged in 5 ml of DMF, and 131 mg (0.68 mmol) of EDC were added. The mixture was stirred at RT for 20 min and then added dropwise to a solution of 221 mg (0.82 mmol) of (2R)-2-amino-2-[3-(trifluoromethyl)phenyl]propionic acid hydrochloride (from Netchem, New Brunswick N.J. 08901, USA, Article No.: 506085-HCl) and 119 μl (0.68 mmol) of N,N-diisopropylethylamine in 2 ml of DMF. The reaction mixture was stirred at RT for 20 min, 1 ml of 1N hydrochloric acid was then added and the complete mixture was purified by preparative HPLC (Method 10). The appropriate fraction was freed from the solvents on a rotary evaporator and the residue was dried under HV. This gave 260 mg (65% of theory) of the title compound.

LC-MS [Method 3]: Rₜ=1.23 min; MS [ESIpos]: m/z=581 (M+H)⁺

¹H-NMR (400 MHz, DMSO-d₆): δ [ppm]=1.85 (s, 3H), 3.76-3.88 (m, 1H), 3.90-4.01 (m, 1H), 4.26 (br.s, 1H), 4.51-4.67 (m, 2H), 6.92 (d, 1H), 7.55-7.71 (m, 4H), 7.71-7.83 (m, 4H), 8.80 (s, 1H), 13.10 (s, 1H).

### Example 6

### (2S)-2-[({3-(4-Chlorophenyl)-5-oxo-4-[(2R)-3,3,3-trifluoro-2-hydroxypropyl]-4,5-dihydro-1H-1,2,4-triazol-1-yl}acetyl)amino]-2-[3-(trifluoromethyl)phenyl]propanamide

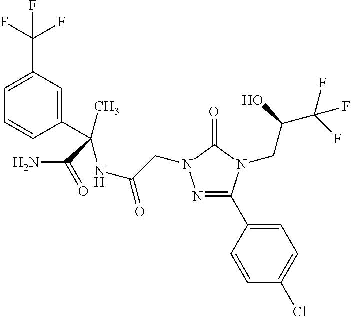

318 mg of the compound from Example 9A (0.87 mmol) were dissolved in 5 ml of DMF, and 250 mg (1.30 mmol) of EDC and 176 mg (1.30 mmol) of HOBt were added. After 30 min of stirring at RT, 269 mg (1 mmol) of the compound from Example 17A and then 303 μl (1.74 mmol) of N,N-diisopropylethylamine were added. The mixture was stirred at RT for 1 h, and the complete mixture was then separated by preparative HPLC (Method 10). The appropriate fraction was freed from the solvents on a rotary evaporator and the residue was dried under HV. This gave 244 mg (47% of theory) of the title compound.

LC-MS [Method 3]: Rₜ=1.22 min; MS [ESIpos]: m/z=580 (M+H)⁺

¹H-NMR (400 MHz, DMSO-d₆): δ [ppm]=1.88 (s, 3H), 3.77-3.87 (dd, 1H), 3.90-4.00 (dd, 1H), 4.26 (m., 1H), 4.52-4.64 (m [AB], 2H), 6.90 (d, 1H), 7.33 (s, 1H), 7.41 (s, 1H), 7.53-7.60 (m, 1H), 7.60-7.67 (m, 3H), 7.68-7.80 (m, 4H), 8.64 (s, 1H).

### Example 7

### 2-({[3-(4-Chlorophenyl)-4-cyclopropyl-5-oxo-4,5-dihydro-1H-1,2,4-triazol-1-yl]acetyl}amino)-2-[3-(trifluoromethyl)phenyl]propanoic acid

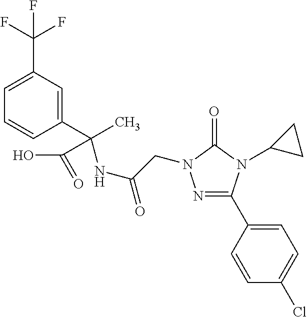

18.2 mg (62 μmol) of [3-(4-chlorophenyl)-4-cyclopropyl-5-oxo-4,5-dihydro-1H-1,2,4-triazol-1-yl]-acetic acid (preparation see WO 2007/134862, Example 88A) were dissolved in 900 μl of DMF, 8.4 mg (62 μmol) of HOBt and then 11.8 mg (62 μmol) of EDC were added and the mixture was stirred at RT for 20 min. 20 mg (74 μmol) of the compound from Example 26A and 22 μl (124 μmol) of N,N-diisopropylethylamine were then added. The mixture was stirred at RT for a further 20 min, and the complete mixture was then separated by preparative HPLC (Method 10). This gave 12 mg (38% of theory) of the title compound.

LC-MS [Method 1]: Rₜ=1.92 min; MS [ESIpos]: m/z=509 (M+H)⁺

¹H-NMR (400 MHz, DMSO-d₆): δ [ppm]=0.51-0.63 (m, 2H), 0.83-0.94 (m, 2H), 1.84 (s, 3H), 3.11-3.22 (m, 1H), 4.51 (s, 2H), 7.55-7.63 (m, 3H), 7.66 (d, 1H), 7.70-7.88 (m, 4H), 8.75 (s, 1H), 13.10 (br.s, 1H).

### Example 8

### 2-({[3-(4-Chlorophenyl)-4-cyclopropyl-5-oxo-4,5-dihydro-1H-1,2,4-triazol-1-yl]acetyl}amino)-2-[3-(trifluoromethyl)phenyl]propanamide

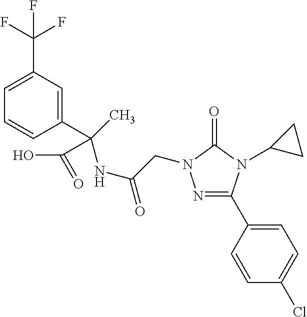

18.0 mg (36 μmol) of the compound from Example 7 were dissolved in 520 ml of DMF, 9.6 mg (71 μmol) of HOBt and then 14 mg (71 μmol) of EDC were added and the mixture was stirred at RT for 20 min. 5 ml of ammonia (35% in water) were then added. The mixture was stirred at RT for a further 20 min, and the complete mixture was then separated by preparative HPLC (Method 10). This gave 9 mg (50% of theory) of the title compound.

LC-MS [Method 6]: Rₜ=2.17 min; MS [ESIpos]: m/z=508 (M+H)⁺

¹H-NMR (400 MHz, DMSO-d₆): δ [ppm]=0.49-0.65 (m, 2H), 0.84-0.93 (m, 2H), 1.87 (s, 3H), 3.17 (dt, 1H), 4.42-4.57 (m [AB], 2H), 7.33 (s, 1H), 7.41 (s, 1H), 7.53-7.65 (m, 4H), 7.67-7.75 (m, 2H), 7.76-7.86 (m, 2H), 8.55 (s, 1H).

### Example 9

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[2-fluoro-3-(trifluoromethyl)phenyl]propanamide (Diastereomer Mixture)

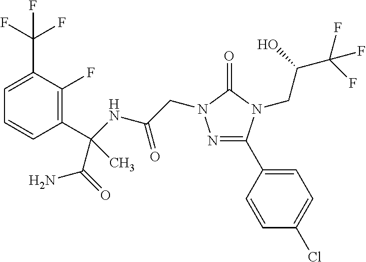

100 mg of the compound from Example 8A (0.27 mmol) were initially charged together with 109 mg (about 0.33 mmol) of the compound from Example 18A, 79 mg (0.41 mmol) of EDC and 55 mg (0.41 mmol) of HOBt in 3 ml of DMF, and 57 μl (0.33 mmol) of N,N-diisopropylethylamine were then added. The mixture was stirred at RT for 1 h, and the complete mixture was then separated by preparative HPLC (Method 10). This gave 90 mg (55% of theory) of the title compound.

LC-MS [Method 3]: Rₜ=1.21 min; MS [ESIpos]: m/z=598 (M+H)⁺

¹H-NMR (400 MHz, DMSO-d₆): δ [ppm]=1.90 (s, 3H), 3.74-3.86 (m, 1H), 3.88-4.00 (m, 1H), 4.25 (br.s, 1H), 4.42-4.58 (m, 2H), 6.89 (d, 1H), 7.22 (s, 1H), 7.38 (t, 1H), 7.44 (s, 1H), 7.59-7.65 (m, 2H), 7.69 (t, 1H), 7.73-7.79 (m, 2H), 7.85 (t, 1H), 8.54 (d, 1H).

The diastereomers from Example 9 could be separated by preparative chromatography on a chiral phase (Method 15): see Example 10 and Example 11.

### Example 10

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[2-fluoro-3-(trifluoromethyl)phenyl]propanamide (Diastereomer I)

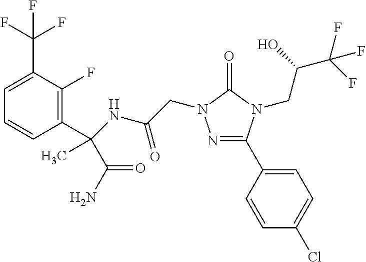

Initially eluted diastereomer (31 mg) from the separation of 85 mg of the compound from Example 9 according to Method 15.

LC-MS [Method 3]: Rₜ=1.21 min; MS [ESIpos]: m/z=598 (M+H)⁺

Analytical chiral HPLC [Method 16]: Rₜ=3.57 min.

¹H-NMR (400 MHz, DMSO-d₆): δ [ppm]=1.90 (s, 3H), 3.74-3.86 (m, 1H), 3.89-4.00 (m, 1H), 4.24 (br.s, 1H), 4.42-4.57 (m [AB], 2H), 6.89 (d, 1H), 7.22 (s, 1H), 7.38 (t, 1H), 7.44 (s, 1H), 7.59-7.65 (m, 2H), 7.69 (t, 1H), 7.72-7.79 (m, 2H), 7.83 (t, 1H), 8.55 (s, 1H).

### Example 11

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[2-fluoro-3-(trifluoromethyl)phenyl]propanamide (Diastereomer II)

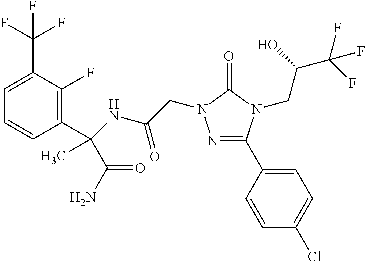

Last eluted diastereomer (30 mg) from the separation of 85 mg of the compound from Example 9 according to Method 15.

LC-MS [Method 3]: Rₜ=1.20 min; MS [ESIpos]: m/z=598 (M+H)⁺

Analytical chiral HPLC [Method 16]: Rₜ=4.19 min.

¹H-NMR (400 MHz, DMSO-d₆): δ [ppm]=1.90 (s, 3H), 3.74-3.86 (m, 1H), 3.89-3.97 (m, 1H), 4.25 (br.s, 1H), 4.42-4.57 (m [AB], 2H), 6.89 (d, 1H), 7.22 (s, 1H), 7.38 (t, 1H), 7.45 (s, 1H), 7.59-7.65 (m, 2H), 7.69 (t, 1H), 7.72-7.79 (m, 2H), 7.85 (t, 1H), 8.54 (s, 1H).

### Example 12

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]butanamide (Diastereomer Mixture)

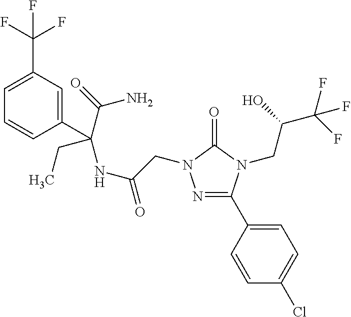

160 mg of the compound from Example 8A (0.44 mmol) were stirred together with 126 mg (0.66 mmol) of EDC and 89 mg (0.66 mmol) of HOBt in 4 ml of DMF for 20, and 136 mg (0.48 mmol) of the compound from Example 19A and 99 μl (0.57 mmol) of N,N-diisopropylethylamine were then added. The mixture was stirred at RT for 2 h, and the complete mixture was then separated by preparative HPLC (Method 10). The appropriate fraction was freed from the solvents on a rotary evaporator and the residue was dried under HV. This gave 59 mg (22% of theory) of the title compound.

LC-MS [Method 3]: Rₜ=1.24 min; MS [ESIpos]: m/z=594 (M+H)⁺

The diastereomers from Example 12 could be separated by preparative chromatography on a chiral phase (Method 17a): see Example 13 and Example 14.

### Example 13

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]butanamide (Diastereomer I)

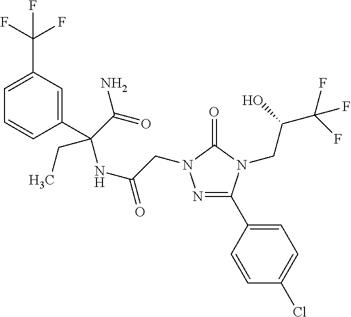

Initially-eluting diastereomer (29 mg) from the separation of 59 mg of the compound from Example 12 according to Method 17a.

Chiral analytical HPLC [Method 18a]: Rₜ=5.2 min.

LC-MS [Method 5]: Rₜ=1.09 min; MS [ESIpos]: m/z=594 (M+H)⁺

¹H-NMR (400 MHz, DMSO-d₆): δ [ppm]=0.79 (t, 3H), 2.42-2.64 (m, 2H), 3.76-3.86 (m, 1H), 3.91-3.99 (m, 1H), 4.25 (br.s, 1H), 4.55-4.66 (m [AB], 2H), 6.89 (d, 1H), 7.40 (d, 2H), 7.52-7.58 (m, 1H), 7.58-7.65 (m, 3H), 7.71 (d, 1H), 7.73-7.80 (m, 3H), 8.43 (s, 1H).

### Example 14

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-[3-(trifluoromethyl)phenyl]butanamide (Diastereomer II)

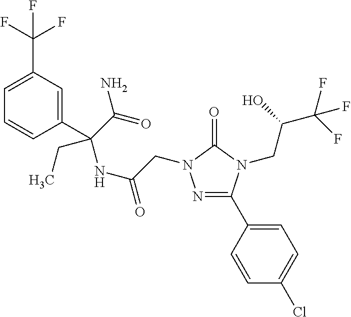

Last-eluting diastereomer (26 mg) from the separation of 59 mg of the compound from Example 12 according to Method 17a.

Chiral analytical HPLC [Method 18a]: Rₜ=9.1 min

LC-MS [Method 5]: Rₜ=1.09 min; MS [ESIpos]: m/z=594 (M+H)⁺

¹H-NMR (400 MHz, DMSO-d₆): δ [ppm]=0.80 (t, 3H), 2.56-2.64 (m, 2H), 3.76-3.87 (m, 1H), 3.89-4.00 (m, 1H), 4.26 (br.s, 1H), 4.61 (s, 2H), 6.89 (d, 1H), 7.40 (d, 2H), 7.52-7.58 (m, 1H), 7.59-7.65 (m, 3H), 7.67-7.78 (m, 4H), 8.44 (s, 1H).

### Example 15

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-2-cyclopropyl-2-[3-(trifluoromethyl)phenyl]acetamide (Diastereomer Mixture)

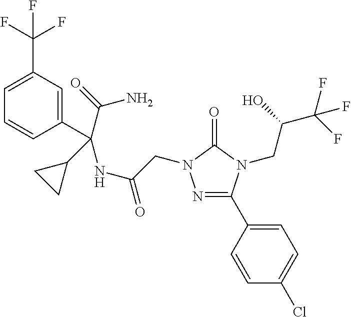

27 mg of the compound from Example 8A (74 μmol) were initially charged together with 24 mg (81 μmol) of the compound from Example 20A, 20 mg (0.10 mmol) of EDC and 14 mg (0.10 mmol) of HOBt in 550 μl of DMF, and 26 μl (0.15 mmol) of N,N-diisopropylethylamine were then added. The mixture was stirred at RT for 2 h, and the complete mixture was then separated by preparative HPLC (Method 10). This gave 24 mg (25% of theory) of the title compound.

LC-MS [Method 3]: Rₜ=1.26 min; MS [ESIpos]: m/z=606 (M+H)⁺

¹H-NMR (400 MHz, DMSO-d₆): δ [ppm]=0.30-0.44 (m, 2H), 0.45-0.62 (m, 2H), 1.80-1.88 (m, 1H), 3.77-3.85 (dd, 1H), 3.91-3.98 (dd, 1H), 4.25 (m., 1H), 4.53-4.61 (m [AB], 2H), 6.89 (2d, 1H), 7.16 (br.s, 1H), 7.34 (s, 1H), 7.49-7.56 (m, 1H), 7.57-7.66 (m, 3H), 7.70-7.80 (m, 3H), 7.87 (br.s, 1H), 8.32 (s, 1H).

### Example 16

### (2R)-2-(2-Chlorophenyl)-2-[({3-(4-chlorophenyl)-5-oxo-4-[(2S)-3,3,3-trifluoro-2-hydroxypropyl]-4,5-dihydro-1H-1,2,4-triazol-1-yl}acetyl)amino]propanoic acid

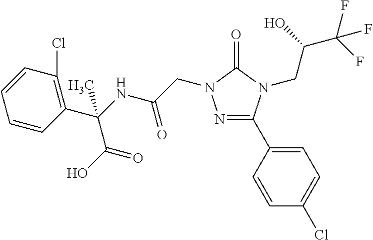

At RT, 134 mg of the compound from Example 8A (0.37 mmol) and 52 mg (0.37 mmol) of HOBt were initially charged in 5 ml of DMF. 70 mg (0.37 mmol) of EDC were added, and the mixture was stirred at RT for 20 min. The solution formed was added dropwise to a suspension of 95 mg (0.40 mmol) of (2R)-2-amino-2-(2-chlorophenyl)propionic acid hydrochloride (from Netchem, New Brunswick N.J. 08901, USA, Article No.: 506093-HCl) and 159 μl (0.91 mmol) of N,N-diisopropylethylamine in 3 ml of DMF, and the resulting mixture was stirred at RT for 4 h. After addition of 2 ml of 1N hydrochloric acid, the complete reaction mixture was separated by preparative HPLC (Method 10). This gave 63 mg (31% of theory) of the title compound.

LC-MS [Method 3]: Rₜ=1.12 min; MS [ESIpos]: m/z=547 (M+H)⁺

¹H-NMR (400 MHz, DMSO-d₆): δ [ppm]=1.96 (s, 3H), 3.71-3.86 (m, 1H), 3.87-3.99 (m, 1H), 4.26 (br.s, 1H), 4.36-4.59 (m [AB], 2H), 6.91 (d, 1H), 7.22-7.39 (m, 3H), 7.55-7.66 (m, 3H), 7.69-7.82 (m, 2H), 8.45 (s, 1H), 13.53 (br.s, 1H).

### Example 17

### (2R)-2-(2-Chlorophenyl)-2-[({3-(4-chlorophenyl)-5-oxo-4-[(2S)-3,3,3-trifluoro-2-hydroxypropyl]-4,5-dihydro-1H-1,2,4-triazol-1-yl}acetyl)amino]propanamide

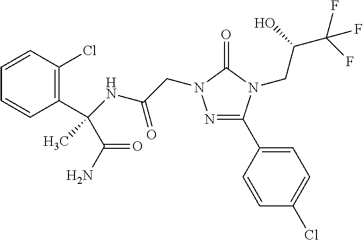

64 mg of the compound from Example 8A (0.17 mmol) together with 45 mg (0.19 mmol) of the compound from Example 22A, 47 mg (0.24 mmol) of EDC and 33 mg (0.24 mmol) of HOBt were initially charged in 2 ml of DMF, and 36 μl (0.21 mmol) of N,N-diisopropylethylamine were then added. The mixture was stirred at RT for 1 h, and the complete mixture was then separated by preparative HPLC (Method 10). This gave 46 mg (48% of theory) of the title compound.

LC-MS [Method 4]: Rₜ=0.96 min; MS [ESIpos]: m/z=546 (M+H)⁺

¹H-NMR (400 MHz, DMSO-d₆): δ [ppm]=1.91 (s, 3H), 3.73-4.00 (m, 2H), 4.26 (br.s, 1H), 4.35-4.55 (m [AB], 2H), 6.80 (s, 1H), 6.91 (d, 1H), 7.24-7.36 (m, 3H), 7.40 (s, 1H), 7.56-7.70 (m, 3H), 7.72-7.83 (m, 2H), 8.37 (s, 1H).

### Example 18

### 2-[({3-(4-Chlorophenyl)-5-oxo-4-[(2S)-3,3,3-trifluoro-2-hydroxypropyl]-4,5-dihydro-1H-1,2,4-triazol-1-yl}acetyl)amino]-3,3,3-trifluoro-2-[3-(trifluoromethyl)phenyl]propanamide (Diastereomer Mixture)

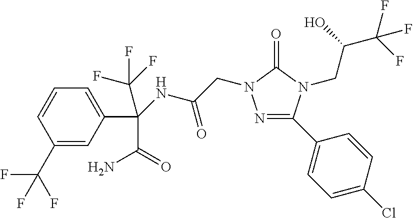

100 mg of the compound from Example 30A (0.21 mmol) were dissolved in 1.5 ml of dichloromethane, and 33.4 mg (0.25 mmol) of Ghosez reagent (1-chloro-N,N,2-trimethylprop-1-ene-1-amine) were added. This solution was stirred at RT for 10 min, and a solution of 65.6 mg (0.23 mmol) of the compound from Example 29A and 27 μl (0.33 mmol) of pyridine in 1.5 ml of dichloromethane was then added. The mixture was stirred at RT for 2 h. Another 72 mg (0.25 mmol) of the compound from Example 29A were added. The mixture was stirred overnight and then freed from the volatile components on a rotary evaporator. To remove the tert-butyl-dimethylsilyl protective group, the residue was taken up in 5 ml of THF, and 0.5 ml of water and then 1 ml of a solution of 1M tetra-n-butylammonium fluoride in THF were added. The resulting solution was stirred at RT for 75 min. The THF was removed on a rotary evaporator and the residue was separated by preparative HPLC (Method 10). This gave 71 mg (53% of theory) of the title compound.

LC-MS [Method 5]: Rₜ=1.12 min; MS [ESIpos]: m/z=634 (M+H)⁺

¹H-NMR (400 MHz, DMSO-d₆): δ [ppm]=3.76-3.88 (m, 1H), 3.91-4.01 (m, 1H), 4.25 (m, 1H), 4.72-4.87 (m, 2H), 6.92 (d, 1H), 7.60-7.82 (m, 8H), 7.91 (d, 1H), 8.00 (s, 1H), 9.43 (s, 1H).

Analytical HPLC on a chiral phase (Method 18a) shows 66% d.e. (initially eluted diastereomer, Rₜ=10 min, 83%; last eluted diastereomer, Rₜ=16 min, 17%).

### Example 19

### (2R)-2-[({3-(4-Chlorophenyl)-5-oxo-4-[(1E)-3,3,3-trifluoroprop-1-en-1-yl]-4,5-dihydro-1H-1,2,4-triazol-1-yl}acetyl)amino]-2-[3-(trifluoromethyl)phenyl]propanamide

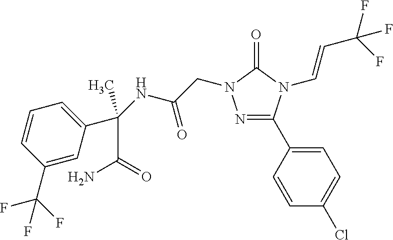

35 mg of the compound from Example 11A (0.10 mmol) together with 32.5 mg (0.12 mmol) of the compound from Example 16A, 23 mg (0.12 mmol) of EDC and 17 mg (0.12 mmol) of HOBt were initially charged in 1.1 ml of DMF, and 26 μl (0.15 mmol) of N,N-diisopropylethylamine were then added. The mixture was stirred at RT for 30 min, 1 ml of 1N hydrochloric acid was then added and the complete mixture was separated by preparative HPLC (Method 10). The appropriate fraction was freed from the solvents on a rotary evaporator and the residue was dried under HV. This gave 55 mg (97% of theory) of the title compound.

LC-MS [Method 4]: Rₜ=1.15 min; MS [ESIpos]: m/z=562 (M+H)⁺

¹H-NMR (400 MHz, DMSO-d₆): δ [ppm]=1.88 (s, 3H), 4.58-4.71 (m [AB], 2H), 6.84 (dq, 1H), 7.16 (dq, 1H), 7.32 (s, 1H), 7.41 (s, 1H), 7.53-7.61 (m, 1H), 7.60-7.70 (m, 5H), 7.71-7.78 (m, 2H), 8.68 (s, 1H).

### Example 20

### 2-[({4-Cyclopropyl-5-oxo-3-[2-(trifluoromethoxy)phenyl]-4,5-dihydro-1H-1,2,4-triazol-1-yl}acetyl)amino]-2-[3-(trifluoromethyl)phenyl]propanamide (Enantiomer Mixture)

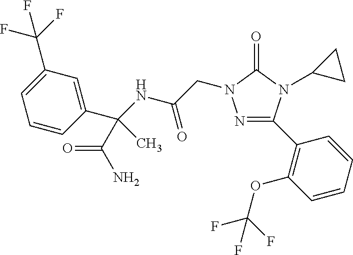

145 mg (0.40 mmol) of the compound from Example 34A were dissolved in 2 ml of DMF, 115 mg (0.60 mmol) of EDC and 81 mg (0.60 mmol) of HOBt were added and the mixture was stirred at room temperature for 10 minutes. 102 mg (0.44 mmol) of the compound from Example 12A were then added, and the mixture was stirred at room temperature for 12 h. The crude mixture was purified directly by preparative HPLC [Method 19]. This gave 136 mg (58% of theory) of the target compound.

LC-MS [Method 1] Rₜ=1.86 min; MS [ESIpos]: m/z=558 (M+H)⁺

¹H-NMR (400 MHz, CDCl₃): δ[ppm]=0.61-0.68 (m, 2H), 0.80-0.87 (m, 2H), 2.01 (s, 3H), 2.97 (spt, 1H), 4.48 and 4.55 (2d, 2H), 5.42 (br.s, 1H), 5.70 (br.s, 1H), 7.39-7.53 (m, 3H), 7.54-7.70 (m, 5H), 7.80 (s, 1H).

The enantiomers from Example 20 could be separated by preparative chromatography on a chiral phase (Method 11): see Example 21 and Example 22

### Example 21

### 2-[({4-Cyclopropyl-5-oxo-3-[2-(trifluoromethoxy)phenyl]-4,5-dihydro-1H-1,2,4-triazol-1-yl}acetyl)amino]-2-[3-(trifluoromethyl)phenyl]propanamide (Enantiomer I)

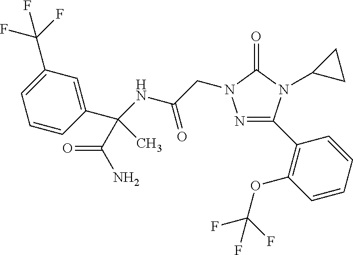

Initially-eluting enantiomer (36 mg) from the separation of 119 mg of the compound from Example 20 according to Method 11.

Analytical chiral HPLC [Method 12]: Rₜ=4.23 min.

### Example 22

### 2-[({4-Cyclopropyl-5-oxo-3-[2-(trifluoromethoxy)phenyl]-4,5-dihydro-1H-1,2,4-triazol-1-yl}acetyl)amino]-2-[3-(trifluoromethyl)phenyl]propanamide (Enantiomer II)

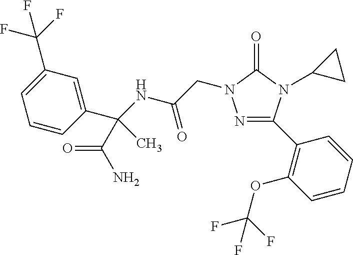

Last-eluting enantiomer (41 mg) from the separation of 119 mg of the compound from Example 20 according to Method 11.

Analytical chiral HPLC [Method 12]: Rₜ=5.04 min.

### Example 23

### 2-({[3-(4-Chlorophenyl)-4-(2-fluorobenzyl)-5-oxo-4,5-dihydro-1H-1,2,4-triazol-1-yl]acetyl}amino)-2-[3-(trifluoromethyl)phenyl]propanamide (Enantiomer Mixture)

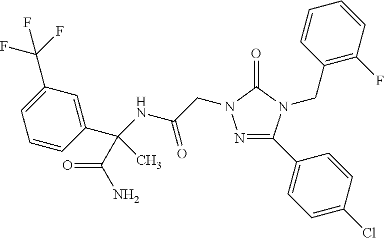

109 mg (0.28 mmol) of [3-(4-chlorophenyl)-4-(2-fluorobenzyl)-5-oxo-4,5-dihydro-1H-1,2,4-triazol-1-yl]acetic acid (preparation see WO 2007/134862, Example 156A) were dissolved in 2 ml of DMF, 79 mg (0.41 mmol) of EDC and 56 mg (0.41 mmol) of HOBt were added and the mixture was stirred at room temperature for 10 minutes. 70 mg (0.30 mmol) of the compound from Example 12A were then added, and the mixture was stirred at room temperature for 20 h. The crude mixture was purified directly by preparative HPLC [Method 19]. This gave 109 mg (69% of theory) of the target compound.

LC-MS [Method 1] Rₜ=2.10 min; MS [ESIpos]: m/z=576 (M+H)⁺

¹H-NMR (400 MHz, CDCl₃): δ[ppm]=2.05 (s, 2H), 4.54 (d, 1H), 4.61 (d, 1H), 5.03 (s, 2H), 5.41 (br.s, 1H), 5.55 (br.s, 1H), 7.03 (t, 1H), 7.09 (t, 1H), 7.14-7.21 (m, 1H), 7.23-7.31 (m, 1H), 7.36-7.45 (m, 4H), 7.46-7.53 (m, 1H), 7.54-7.66 (m, 2H), 7.69 (s, 1H), 7.97 (s, 1H).

The enantiomers from Example 23 could be separated by preparative chromatography on a chiral phase (Method 17a): see Example 24 and Example 25.

### Example 24

### 2-({[3-(4-Chlorophenyl)-4-(2-fluorobenzyl)-5-oxo-4,5-dihydro-1H-1,2,4-triazol-1-yl]acetyl}amino)-2-[3-(trifluoromethyl)phenyl]propanamide (Enantiomer I)

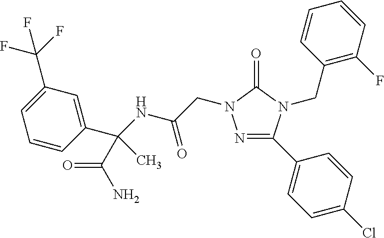

Initially-eluting enantiomer (11 mg) from the separation of 108 mg of the compound from Example 23 according to Method 17a.

Analytical chiral HPLC [Method 18a]: Rₜ=2.12 min.

### Example 25

### 2-({[3-(4-Chlorophenyl)-4-(2-fluorobenzyl)-5-oxo-4,5-dihydro-1H-1,2,4-triazol-1-yl]acetyl}amino)-2-[3-(trifluoromethyl)phenyl]propanamide (Enantiomer II)

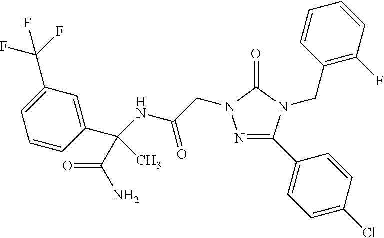

Last-eluting enantiomer (31 mg) from the separation of 108 mg of the compound from Example 23 according to Method 17a.

Analytical chiral HPLC [Method 18a]: Rₜ=2.48 min.

### B. EVALUATION OF THE PHARMACOLOGICAL ACTIVITY

The pharmacological action of the compounds according to the invention can be shown in the following assays:

### Abbreviations:

EDTA ethylenediaminetetraacetic acid

### DMEM Dulbecco's Modified Eagle Medium

FCS fetal calf serum
HEPES 4-(2-hydroxyethyl)-1-piperazineethanesulfonic acid

### SmGM Smooth Muscle Cell Growth Media

Tris-HCl 2-amino-2-(hydroxymethyl)-1,3-propanediol hydrochloride

### UtSMC Uterine Smooth Muscle Cells

### B-1. Cellular In Vitro Assay for Determining the Vasopressin Receptor Activity

The identification of agonists and antagonists of the V1a and V2 vasopressin receptors from humans and rats and also the quantification of the activity of the substances described here took place using recombinant cell lines. These cells derive originally from a hamster ovary epithelial cell (Chinese Hamster Ovary, CHO K1, ATCC: American Type Culture Collection, Manassas, Va. 20108, USA). The test cell lines constitutively express a modified form of the calcium-sensitive photoprotein aequorin, which, after reconstitution with the cofactor coelenterazine, emits light when there are increases in the free calcium concentration (Rizzuto R., Simpson A. W., Brini M., Pozzan T.; Nature 358 (1992) 325-327). In addition, the cells are stably transfected with the human or rat V1a or V2 receptors. In the case of the Gs-coupling V2 receptors, the cells are stably transfected with a further gene, which codes for the promiscuous G_{α16} protein (Amatruda T. T., Steele D. A., Slepak V. Z., Simon M. I., Proc. Nat. Acad. Sci. USA 88 (1991), 5587-5591), either independently or as a fusion gene. The resulting vasopressin receptor test cells react to stimulation of the recombinantly expressed vasopressin receptors by intracellular release of calcium ions, which can be quantified by the resulting aequorin luminescence using a suitable luminometer (Milligan G., Marshall F., Rees S., Trends in Pharmaco. Sci. 17 (1996) 235-237).

Test procedure: On the day before the assay, the cells are plated out in culture medium (DMEM, 10% FCS, 2 mM glutamine, 10 mM HEPES) in 384-well microtiter plates and kept in a cell incubator (96% humidity, 5% v/v carbon dioxide, 37° C.). On the day of the assay, the culture medium is replaced by a Tyrode solution (140 mM sodium chloride, 5 mM potassium chloride, 1 mM magnesium chloride, 2 mM calcium chloride, 20 mM glucose, 20 mM HEPES), which additionally contains the cofactor coelenterazine (50 μM), and the microtiter plate is then incubated for a further 3-4 hours. The test substances in various concentrations are placed for 10 to 20 minutes in the wells of the microtiter plate before the agonist [Arg8]-vasopressin is added, and the resulting light signal is measured immediately in the luminometer. The IC50 values are calculated using the GraphPad PRISM computer program (Version 3.02).

The table below lists representative IC₅₀ values for the compounds of the invention on the cell line transfected with the human V1a or V2 receptor:

TABLE 1

| Example No. | IC₅₀ hV1a [μM] | IC₅₀ hV2 [μM] |
|---|---|---|
| 1 | 0.118 | 0.012 |
| 3 | 0.106 | 0.028 |
| 4 | 0.0022 | 0.0037 |
| 14 | 0.006 | 0.0064 |
| 17 | 0.012 | 0.22 |
| 19 | 0.013 | 0.014 |
| 25 | 0.006 | 0.016 |

### B-2. Cellular In Vitro Assay for Detecting the Action of Vasopressin V1a Receptor Antagonists on the Regulation of Pro-Fibrotic Genes

The cell line H9C2 described as of cardiomyocyte type (American Type Culture Collection ATCC No. CRL-1446), isolated from rat cardiac tissue, endogenously expresses the vasopressin V1A receptor AVPR1A in high copy number, whereas the AVPR2 expression cannot be detected. For cell assays on the inhibition of the AVPR1A receptor-dependent regulation of gene expression by receptor antagonists, the procedure is as follows:

H9C2 cells are seeded in 12-well microtiter plates for cell culture, at a cell density of 100 000 cells/well, in 1.0 ml of Opti-MEM medium (Invitrogen Corp. Carlsbad Calif., USA, Cat. No. 11058-021) with 2% FCS and 1% penicillin/streptomycin solution (Invitrogen Cat. No. 10378-016), and held in a cell incubator (96% humidity, 5% v/v carbon dioxide, 37° C.). After 24 hours, sets of three wells (triplicate) are charged with vehicle solution (negative control), vasopressin solution: [Arg8]-vasopressin acetate (Sigma Cat. No. V9879) or test substances (dissolved in vehicle: water with 20% by volume ethanol) and vasopressin solution. In the cell culture, the final vasopressin concentration is 0.05 μM. The test substance solution is added to the cell culture in small volumes, and so a final concentration of 0.1% of ethanol in the cell assay is not exceeded. After an incubation time of 6 hours, the culture supernatant is drawn off under suction, the adherent cells are lysed in 250 μl of RLT buffer (Qiagen, Ratingen, Cat. No. 79216), and the RNA is isolated from this lysate using the RNeasy kit (Qiagen, Cat. No. 74104). This is followed by DNAse digestion (Invitrogen Cat. No. 18068-015), cDNA synthesis (Promaga ImProm-II Reverse Transcription System Cat. No. A3800) and RTPCR using the pPCR MasterMix RT-QP2X-03-075 from Eurogentec, Seraing, Belgium. All procedures take place in accordance with the working protocols of the test reagents' manufacturers. The primer sets for the RTPCR are selected on the basis of the mRNA gene sequences (NCBI Genbank Entrez Nucleotide Data Base) using the Primer3Plus program with 6-FAM-TAMRA labelled probes. The RTPCR for determining the relative mRNA expression in the cells of the various assay batches is carried out using the Applied Biosystems ABI Prism 7700 Sequence Detector in 96-well or 384-well microtiter plate format in accordance with the instrument operating instructions. The relative gene expression is represented by the delta-delta Ct value [Applied Biosystems, User Bulletin No. 2 ABI Prism 7700 SDS Dec. 11, 1997 (updated 10/2001)] with reference to the level of expression of the ribosomal protein L-32 gene (Genbank Acc. No. NM_{—}013226) and the threshold Ct value of Ct=35.

### B-3. In Vivo Test for Detection of Cardiovascular Effect: Blood Pressure Measurement on Anaesthetised Rats (Vasopressin ‘Challenge’ Model)

In male Sprague-Dawley rats (250-350 g body weight) under ketamine/xylazine/pentobarbital injection anaesthesia, polyethylene tubes (PE-50; Intramedic®), which are prefilled with heparin-containing (500 IU/ml) isotonic sodium chloride solution, are introduced into the jugular vein and the femoral vein and then tied in. Via one venous access, with the aid of a syringe, arginine-vasopressin is injected; the test substances are administered via the second venous access. For determination of the systolic blood pressure, a pressure catheter (Millar SPR-320 2F) is tied into the carotid artery. The arterial catheter is connected to a pressure transducer which feeds its signals to a recording computer equipped with suitable recording software. In a typical experiment the experimental animal is administered 3-4 successive bolus injections at intervals of 10-15 min with a defined amount of arginine-vasopressin (30 ng/kg) in isotonic sodium chloride solution and, when the blood pressure has reached initial levels again, the substance under test is administered as a bolus, with subsequent ongoing infusion, in a suitable solvent. After this, at defined intervals (10-15 min), the same amount of vasopressin as at the start is administered again. On the basis of the blood pressure values, a determination is made of the extent to which the test substance counteracts the hypertensive effect of the vasopressin. Control animals receive only solvent instead of the test substance.

Following intravenous administration, the compounds of the invention, in comparison to the solvent controls, bring about an inhibition in the blood pressure increase caused by arginine-vasopressin.

### B-4. In Vivo Assay for Detecting the Cardiovascular Effect: Diuresis Investigations on Conscious Rats in Metabolism Cages

Wistar rats (220-400 g body weight) are kept with free access to feed (Altromin) and drinking water. During the experiment, the animals are kept with free access to drinking water for 4 to 8 hours individually in metabolism cages suitable for rats of this weight class (Tecniplast Deutschland GmbH, D-82383 Hohenpeiβenberg). At the beginning of the experiment, the animals are administered the substance under test in a volume of 1 to 3 ml/kg body weight of a suitable solvent by means of gavage into the stomach. Control animals receive only solvent. Controls and substance tests are carried out in parallel on the same day. Control groups and substance-dose groups each consist of 4 to 8 animals. During the experiment, the urine excreted by the animals is collected continuously in a receiver at the base of the cage. The volume of urine per unit time is determined separately for each animal, and the concentration of the sodium and potassium ions excreted in the urine is measured by standard methods of flame photometry. To obtain a sufficient volume of urine, the animals are given a defined amount of water by gavage at the beginning of the experiment (typically 10 ml per kilogram of body weight). Before the beginning of the experiment and after the end of the experiment, the body weight of the individual animals is taken.

Following oral administration, in comparison with control animals, the compounds of the invention bring about an increased excretion of urine, which is based essentially on an increased excretion of water (aquaresis).

### B-5. In Vivo Assay for Detecting the Cardiovascular Effect: Haemodynamic Investigations on Anaesthetised Dogs

Male or female mongrel dogs (Mongrels, Marshall BioResources, USA) with a weight of between 20 and 30 kg are anaesthetised with pentobarbital (30 mg/kg iv, Narcoren®, Merial, Germany) for the surgical interventions and the haemodynamic and functional investigation termini. Alcuronium chloride (Alloferin®, ICN Pharmaceuticals, Germany, 3 mg/animal iv) serves additionally as a muscle relaxant. The dogs are intubated and ventilated with an oxygen/ambient air mixture (40/60%) (about 5-6 L/min). Ventilation takes place using a ventilator from Draeger (Sulla 808) and is monitored using a carbon dioxide analyser (Engström).

The anaesthesia is maintained by continual infusion of pentobarbital (50 μg/kg/min); fentanyl is used as an analgesic (10 μg/kg/h). One alternative to pentobarbital is to use isoflurane (1-2% by volume).

In preparatory interventions, the dogs are fitted with a cardiac pacemaker.
At a time of 21 days before the first drug testing (i.e. start of experiment), a cardiac pacemaker from Biotronik (Logos®) is implanted into a subcutaneous skin pocket and is contacted with the heart via a pacemaker electrode which is advanced through the external jugular vein, with illumination, into the right ventricle.
At the same time as the implanting of the pacemaker, through retrograde advancing of a 7F biopsy forceps (Cordis) via a sheath introducer (Avanti+®; Cordis) in the fermoral artery, and after atraumatic passage through the aortic valve, there is defined lesion of the mitral valve, with monitoring by echo cardiography and illumination. Thereafter all of the accesses are removed and the dog wakes spontaneously from the anaesthesia.
After a further 7 days (i.e. 14 days before the first drug testing), the above pacemaker is activated and the heart is stimulated at a frequency of 220 beats per minute.

The actual drug testing experiments take place 14 and 28 days after the beginning of pacemaker stimulation, using the following instrumentation:
Bladder catheter for bladder relief and for measuring the flow of urine
ECG leads to the extremities (for ECG measurement)
Introduction of an NaCl-filled Fluidmedic PE-300 tube into the femoral artery. This tube is connected to a pressure sensor (Braun Melsungen, Melsungen, Germany) for measuring the systemic blood pressure
Introduction of a Millar Tip catheter (type 350 PC, Millar Instruments, Houston, USA) through the left atrium or through a port secured in the carotid artery, for measuring cardiac haemodynamics
Introduction of a Swan-Ganz catheter (CCOmbo 7.5F, Edwards, Irvine, USA) via the jugular vein into the pulmonary artery, for measuring the cardiac output, oxygen saturation, pulmonary arterial pressures and central venous pressure
Siting of a Braunüle in the cephalic vein, for infusing pentobarbital, for liquid replacement and for blood sampling (determination of the plasma levels of substance or other clinical blood values)
Siting of a Braunüle in the saphenous vein, for infusing fentanyl and for administration of substance
Infusion of vasopressin (Sigma) in increasing dosage, up to a dose of 4 mU/kg/min. The pharmacological substances are then tested with this dosage.

The primary signals are amplified if necessary (Gould amplifier, Gould Instrument Systems, Valley View, USA) or Edwards-Vigilance-Monitor (Edwards, Irvine, USA) and subsequently fed into the Ponemah system (DataSciences Inc, Minneapolis, USA) for evaluation. The signals are recorded continuously throughout the experimental period, and are further processed digitally by said software, and averaged over 30 s.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% strength solution (m/m) of the PVP in water. After drying, the granules are mixed with the magnesium stearate for 5 minutes. This mixture is compressed using a conventional tableting press (for tablet format see above). The guideline compressive force used for compression is 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

A single dose of 100 mg of the compound of the invention is given by 10 ml of oral suspension.

### Production:

The Rhodigel is suspended in ethanol, and the compound of the invention is added to the suspension. The water is added with stirring. Stirring is continued for about 6 h until the swelling of the Rhodigel is ended.

### Solution for Oral Administration:

### Composition:

500 mg of the compound of the invention, 2.5 g of polysorbate and 97 g of polyethylene glycol 400. A single dose of 100 mg of the compound of the invention is given by 20 g of oral solution.

### Production:

The compound of the invention is suspended with stirring in the mixture of polyethylene glycol and polysorbate. The stirring operation continues until the compound of the invention is fully dissolved.

### I.V. Solution:

The compound of the invention is dissolved at a concentration below saturation solubility in a physiologically tolerated solvent (e.g. isotonic saline solution, 5% glucose solution and/or 30% PEG 400 solution). The solution is sterile-filtered and dispensed into sterile, pyrogen-free injection containers.

1. A compound of formula (I) 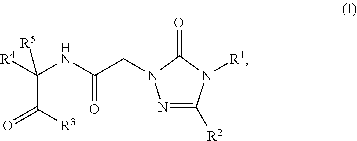
in which
R¹ represents (C₁-C₆)-alkyl, (C₂-C₆)-alkenyl, (C₂-C₆)-alkynyl or (C₃-C₇)-cycloalkyl,
where (C₁-C₆)-alkyl, (C₂-C₆)-alkenyl and (C₂-C₆)-alkynyl may be substituted by 1 to 3 substituents independently of one another selected from the group consisting of halogen, cyano, oxo, hydroxyl, trifluoromethyl, (C₃-C₇)-cycloalkyl, (C₁-C₆)-alkoxy, trifluoromethoxy and phenyl,
where (C₃-C₇)-cycloalkyl may be substituted by 1 or 2 substituents independently of one another selected from the group consisting of (C₁-C₄)-alkyl, oxo, hydroxyl, (C₁-C₄)-alkoxy and amino
and
where (C₁-C₆)-alkoxy may be substituted by 1 or 2 substituents independently of one another selected from the group consisting of amino, hydroxyl, (C₁-C₄)-alkoxy, hydroxycarbonyl and (C₁-C₄)-alkoxycarbonyl
and
where phenyl may be substituted by 1 to 3 substituents independently of one another selected from the group consisting of halogen, cyano, nitro, (C₁-C₄)-alkyl, trifluoromethyl, hydroxyl, hydroxymethyl, (C₁-C₄)-alkoxy, trifluoromethoxy, (C₁-C₄)-alkoxymethyl, hydroxycarbonyl and (C₁-C₄)-alkoxycarbonyl,
and
where (C₃-C₇)-cycloalkyl may be substituted by 1 or 2 substituents independently of one another selected from the group consisting of fluorine, (C₁-C₄)-alkyl, (C₁-C₄)-alkoxy, hydroxyl, amino and oxo,
R² represents phenyl, naphthyl, thienyl, benzothienyl, furyl or benzofuryl,
where phenyl, naphthyl, thienyl, benzothienyl, furyl and benzofuryl may be substituted by 1 to 3 substituents independently of one another selected from the group consisting of halogen, cyano, nitro, (C₁-C₄)-alkyl, trifluoromethyl, hydroxyl, (C₁-C₄)-alkoxy, trifluoromethoxy and phenyl,
where phenyl may be substituted by 1 or 2 substituents independently of one another selected from the group consisting of halogen, cyano, nitro, (C₁-C₄)-alkyl, trifluoromethyl, hydroxyl, (C₁-C₄)-alkoxy, trifluoromethoxy, hydroxy-(C₁-C₄)-alkyl and (C₁-C₄)-alkylthio,
R³ represents hydroxyl or —NR⁶R⁷,
where
R⁶ represents hydrogen or (C₁-C₄)-alkyl,
R⁷ represents hydrogen, (C₁-C₄)-alkyl or (C₃-C₇)-cycloalkyl,
R⁴ represents phenyl,
where phenyl may be substituted by 1 to 3 substituents independently of one another selected from the group consisting of halogen, cyano, nitro, (C₁-C₄)-alkyl, difluoromethyl, trifluoromethyl, hydroxyl, (C₁-C₄)-alkoxy, difluoromethoxy, trifluoromethoxy and phenyl,
where phenyl may be substituted by 1 or 2 substituents independently of one another selected from the group consisting of halogen, cyano, nitro, (C₁-C₄)-alkyl, trifluoromethyl, hydroxyl, (C₁-C₄)-alkoxy, trifluoromethoxy, hydroxy-(C₁-C₄)-alkyl and (C₁-C₄)-alkylthio,
R⁵ represents trifluoromethyl, (C₁-C₄)-alkyl or (C₃-C₇)-cycloalkyl,
or a salt, a solvate or a solvate of a salt thereof.

2. The compound of claim 1 in which
R¹ represents (C₁-C₆)-alkyl, (C₂-C₆)-alkenyl or (C₃-C₆)-cycloalkyl,
where (C₁-C₆)-alkyl and (C₂-C₆)-alkenyl may be substituted by 1 to 3 substituents independently of one another selected from the group consisting of fluorine, chlorine, cyano, oxo, hydroxyl, trifluoromethyl, cyclopropyl, cyclobutyl, methoxy, ethoxy, trifluoromethoxy and phenyl,
where phenyl may be substituted by 1 to 3 substituents independently of one another selected from the group consisting of fluorine, chlorine, cyano, methyl, ethyl, trifluoromethyl, hydroxyl, hydroxymethyl, methoxy, ethoxy, trifluoromethoxy, methoxymethyl, ethoxymethyl, hydroxycarbonyl, methoxycarbonyl and ethoxycarbonyl,
and
where (C₃-C₆)-cycloalkyl may be substituted by 1 or 2 substituents independently of one another selected from the group consisting of fluorine, methyl, ethyl, methoxy, ethoxy, hydroxyl, amino and oxo,
R² represents phenyl or thienyl,
where phenyl and thienyl may be substituted by 1 or 2 substituents independently of one another selected from the group consisting of fluorine, chlorine, cyano, methyl, ethyl, trifluoromethyl, hydroxyl, methoxy, ethoxy and trifluormethoxy,
R³ represents hydroxyl or —NR⁶R⁷,
where
R⁶ represents hydrogen or (C₁-C₄)-alkyl,
R⁷ represents hydrogen, (C₁-C₄)-alkyl or (C₃-C₅)-cycloalkyl,
R⁴ represents phenyl,
where phenyl may be substituted by 1 to 3 substituents independently of one another selected from the group consisting of fluorine, chlorine, cyano, methyl, ethyl, difluoromethyl, trifluoromethyl, methoxy, ethoxy, difluoromethoxy and trifluoromethoxy,
R⁵ represents trifluoromethyl, methyl, ethyl, isopropyl or cyclopropyl,
or a salt, a solvate or a solvate of a salt thereof.

3. The compound of claim 1 in which
R¹ represents (C₁-C₆)-alkyl, (C₂-C₆)-alkenyl or cyclopropyl,
where (C₁-C₆)-alkyl and (C₂-C₆)-alkenyl may be substituted by 1 or 2 substituents independently of one another selected from the group consisting of fluorine, oxo, hydroxyl, trifluoromethyl, cyclopropyl and phenyl,
where phenyl may be substituted by 1 or 2 substituents independently of one another selected from the group consisting of fluorine, chlorine, methyl and methoxy,
R² represents phenyl,
where phenyl may be substituted by 1 or 2 substituents independently of one another selected from the group consisting of fluorine, chlorine, methyl, methoxy and trifluoromethoxy,
R³ represents hydroxyl or —NR⁶R⁷,
where
R⁶ represents hydrogen or methyl,
R⁷ represents hydrogen, methyl or cyclopropyl,
R⁴ represents phenyl,
where phenyl may be substituted by 1 or 2 substituents independently of one another selected from the group consisting of fluorine, chlorine, methyl, trifluoromethyl, methoxy and trifluoromethoxy,
R⁵ represents methyl or ethyl,
or a salt, a solvate or a solvate of a salt thereof.

4. A process for preparing compounds of claim 1 wherein
[A] a compound of the formula (II) 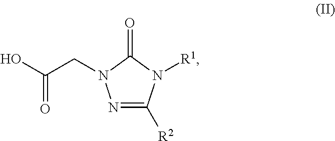
in which R¹ and R² each have the meanings given in claim 1
is coupled in an inert solvent with activation of the carboxylic acid function with a compound of the formula (III) 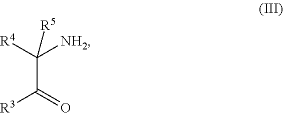
in which R³, R⁴ and R⁵ each have the meanings given in claim 1,
or
[B] a compound of the formula (IV) 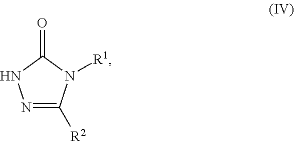
in which R¹ and R² each have the meanings given in claim 1,
is reacted in an inert solvent in the presence of a base with a compound of the formula (V) 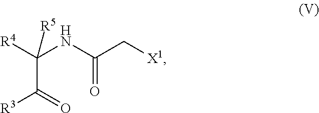
in which R³, R⁴ and R⁵ each have the meanings given in claim 1
and
X¹ represents a leaving group such as, for example, halogen, mesylate or tosylate,
or
[C] a compound of the formula (I-A) 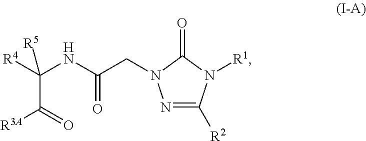
in which R¹, R², R⁴ and R⁵ each have the meanings given in claim 1
and
R^{3A} represents hydroxyl,
is reacted in an inert solvent with activation of the carboxylic acid function with an amine of the formula (VI) 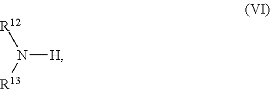
in which R¹² and R¹³ each have the meanings given in claim 1
and the resulting compounds of the formula (I) are optionally converted with the appropriate (i) solvents and/or (ii) bases or acids into their solvates, salts and/or solvates of the salts.

5. (canceled)

6. (canceled)

7. (canceled)

8. A pharmaceutical composition comprising a compound of claim 1 and an inert non-toxic pharmaceutically suitable auxiliary.

9. The pharmaceutical composition of claim 1, further comprising an active substances selected from the group consisting of a diuretic, an angiotensin AII antagonist, an ACE inhibitor, a beta-receptor blocker, a mineralocorticoid receptor antagonist, an organic nitrate, an NO donator and a positive-inotropic active substance.

10. (canceled)

11. A method for the treatment and/or prophylaxis of acute and chronic heart failure, hypervolemic and euvolemic hyponatremia, cirrhosis of the liver, ascites, edema and the syndrome of inadequate ADH secretion (SIADH) comprising administering to a patient in need thereof an effective amount of at least one compound of claim 1.
